(19) United States
(12) Patent Application Publication
(10) Pub. No.: US 2005/0031597 A1
(43) Pub. Date: Feb. 10, 2005

(54) Cells and improved method for preserving cells

(76) Inventors: John H. Crowe, Davis, CA (US); Fern Tablin, Davis, CA (US); Nelly M. Tsvetkova, Davis, CA (US); Zsolt Torok, Davis, CA (US); Gyana R. Satpathy, Davis, CA (US); Rachna Bali, West Sacramento, CA (US); Denis M. Dwyre, Iowa City, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/724,372

(22) Filed: Nov. 28, 2003

Related U.S. Application Data
(63) Continuation-in-part of application No. 10/635,795, filed on Aug. 6, 2003.

(51) Int. Cl. A61K 45/00; A01N 1/02; A61K 31/70; A61K 31/045

(52) U.S. Cl. 424/93.7; 435/2; 514/23; 514/46; 514/738

(57) ABSTRACT

A method for maintaining a viability level of a metabolite in a biological sample comprising disposing a biological sample into a solute solution comprising a solute and a chemical selected from the group consisting of a monosaccharide, a monosaccharide polyol, a cell metabolite-controlling agent, a salt, and mixtures thereof; and incubating the biological sample in the solute solution while maintaining a viability level of a metabolite in the biological sample.

### RELATED PATENT APPLICATIONS

This patent application is a continuation-in-part patent application of copending patent application having Ser. No. 10,635,795, filed Aug. 6, 2003, fully incorporated herein by reference thereto as if repeated verbatim immediately hereinafter. This patent application is related to co-pending patent application Ser. No. 10/052,162, filed Jan. 16, 2002. Patent application Ser. No. 10/052,162 is a continuation-in-part patent application of co-pending patent application Ser. No. 09/927,760, filed Aug. 9, 2001. Patent application Ser. No. 09/927,760 is a continuation-in-part patent application of co-pending patent application Ser. No. 09/828,627, filed Apr. 5, 2001. Patent application Ser. No. 09/828,627 is a continuation patent application of patent application Ser. No. 09/501,773, filed Feb. 10, 2000. All of the foregoing patent applications are fully incorporated herein by reference thereto as if repeated verbatim immediately hereinafter.

### STATEMENT REGARDING FEDERAL SPONSORED RESEARCH AND DEVELOPMENT

Embodiments of this invention were made with Government support under Grant No. N66001-00-C-8048, awarded by the Department of Defense Advanced Research Projects Agency (DARPA). Further embodiments of this invention were made with Government support under Grant Nos. HL57810 and HL61204, awarded by the National Institutes of Health. The Government has certain rights to embodiments of this invention.

### FIELD OF THE INVENTION

Embodiments of the present invention generally broadly relate to living mammalian cells. More specifically, embodiments of the present invention generally provide for the preservation and survival of cells, especially human cells, such as erythrocytic cells.

Embodiments of the present invention also generally broadly relate to the therapeutic uses of cells; and more particularly to manipulations or modifications of erythrocytic cells, such as loading erythrocytic cells with solutes and in preparing freeze-dried compositions that can be re-hydrated at the time of application. When cells for various embodiments of the present invention are re-hydrated, they are immediately restored to viability.

The compositions and methods for embodiments of the present invention are useful in many applications, such as in medicine, pharmaceuticals, biotechnology, and agriculture, and including transfusion therapy, as hemostasis aids and for drug delivery.

### BACKGROUND OF THE INVENTION

A cell is broadly regarded in the art as a small, typically microscopic, mass of protoplasm bounded externally by a semi-permeable membrane, usually including one or more nuclei and various other organelles with their products. A cell is capable either alone or interacting with other cells of performing all the fundamental function(s) of life, and forming the smallest structural unit of living matter capable of functioning independently.

Cells may be transported and transplanted; however, this requires cryopreservation which includes freezing and subsequent reconstitution (e.g., thawing, re-hydration, etc.) after transportation. Unfortunately, a very low percentage of cells retain their functionality after undergoing freezing and thawing. While some cryoprotectants, such as dimethyl sulfoxide, tend to lessen the damage to cells, they still do not prevent some loss of cell functionality.

Trehalose has been found to be suitable in the cryopreservation of cells and platelets. Trehalose is a disaccharide found at high concentrations in a wide variety of organisms that are capable of surviving almost complete dehydration. Trehalose has been shown to stabilize membranes, proteins, and certain cells during freezing and drying in vitro.

U.S. Pat. No. 5,827,741, Beattie et al., issued Oct. 27, 1998, discloses cryoprotectants for human cells and platelets, such as dimethylsulfoxide and trehalose. The cells or platelets may be suspended, for example, in a solution containing a cryoprotectant at a temperature of about 22° C. and then cooled to below 15° C. This incorporates some cryoprotectant into the cells or platelets, but not enough to prevent hemolysis of a large percentage of the cells or platlets.

Accordingly, a need exists for the effective and efficient preservation of cells. More specifically, and accordingly further, a need also exists for the effective and efficient cryopreservation of cells (e.g., erythrocytic cells, eukaryotic cells, or any other cells, and the like), such that the preserved cells respectively maintain their biological properties and may readily become viable after storage.

### SUMMARY OF EMBODIMENTS OF THE INVENTION

In one aspect of the present invention, a dehydrated composition is provided having a generally dehydrated composition comprising freeze-dried cells selected from a mammalian species (e.g., a human) and being effectively loaded internally (e.g., producing hyper-osmotic pressure on the cells to uptake external trehalose via fluid phase endocytosis) with at least about 10 mM of a carbohydrate (e.g., an oligosaccharide, such as trehalose) therein to preserve biological properties during freeze-drying and re-hydration. The amount of the carbohydrate inside the freeze-dried cells is preferably the amount obtained from maintaining a positive loading gradient or loading efficiency gradient on the cell. When the carbohydrate is trehalose, the amount of trehalose loaded inside the freeze-dried cells is preferably from about 10 mM to about 50 mM.

In another aspect of the present invention, a method is provided for loading (e.g., by fluid phase endocytosis) a solute into a cell (e.g., an erythrocytic cell). Embodiments of the invention include disposing a cell in a solution having a solute concentration of sufficient magnitude to produce hyper-osmotic pressure on the cell for transferring a solute (e.g., an oligosaccharide, such as trehalose) from the solution into the cell. The method may additionally comprise preventing a decrease in a loading efficiency gradient in the loading of the solute into the cell. In an embodiment of the invention where the solute comprises an oligosaccharide, the preventing a decrease in a loading efficiency gradient in the loading of the oligosaccharide into the cell may comprise maintaining a concentration of the oligosaccharide in the oligosaccharide solution below a certain concentration, such as below from about 35 mM to about 65 mM, more particularly below a concentration ranging from about 40 mM to about 60 mM, more particularly further below a concentration ranging from about 45 mM to about 55 mM (e.g., below about 50 mM). In another embodiment of the invention, the preventing a decrease in a loading efficiency gradient in the loading of the oligosaccharide into the cell comprises maintaining a positive gradient of loading efficiency to concentration of the oligosaccharide in the oligosaccharide solution.

The solute concentration includes an extracellular cellular solute concentration for elevating extracellular osmolarity within the solution to a value which is greater than a value of the intracellular osmolarity of the cell. The transferring of the solute is preferably by fluid phase endocytosis and preferably without degradation of the solute. In embodiments of the invention where the cell is an erythrocytic cell and the solute comprises trehalose, a gradient of trehalose (mM) within the erythrocytic cell to extracellular trehalose concentration (mM) within the solution may range from about 0.130 to about 0.200, particularly for a temperature ranging from about 30° C. to about 40° C. (e.g., about 37° C.). In a further embodiment of the invention, a gradient of trehalose (mM) within the erythrocytic cell to extracellular trehalose concentration (mM) within the solution ranges from about 0.04 to about 0.12, particularly for a temperature ranging from about 0° C. to about 10° C. In yet a further embodiment, a gradient of trehalose (mM) within the erythrocytic cell to extracellular trehalose concentration (mM) within the solution may range from about 0.04 to about 0.08, or from about 0.08 to bout 0.12, particularly for a temperature ranging from about 0° C. to about 10° C. The solute solution may have a trehalose concentration ranging from about 320 mM to about 4000 mM, such as including from about 320 mM to about 2000 mM or from about 500 mM to about 1000 mM.

A further embodiment of the invention provides retaining the solute in the cell; more specifically, washing the cell and retaining the solute in the cell during the washing. The washing is with a washing buffer, and retention of the solute in the cell increases from about 25% to about 175% when a buffer concentration (e.g., the osmolarity of all osmotically active particles within the washing buffer solution) increases from about 50% to about 400%, more preferably from about 50% to about 150% when a buffer concentration increases from about 100% to about 300%, and most preferably from about 75% to about 125% (e.g., about 100%) when a buffer concentration increases from about 150% to about 250% (e.g., about 200%). The washing of the cell with a washing buffer includes employing a ratio of an extracellular buffer concentration (mOsm) to an intracellular solute concentration (mM) ranging from about 14.0 to about 4.0, such as from about 12.0 to about 5.0, including from about 9.0 to about 6.0 and from about 8.0 to about 7.0 (e.g., about 7.5).

Additional embodiments of the present invention provide a method for loading trehalose into an erythrocytic cell. The method may comprise disposing an erythrocytic cell in a trehalose solution having a trehalose concentration of at least about 25% (preferably at least about 50%) greater than the intracellular osmolarity of the erythrocytic cell for loading (e.g., by fluid phase endocytosis) the trehalose into the erythrocytic cell.

The loading of the trehalose from the trehalose solution into the erythrocytic cell may be without degradation of the trehalose, and produces a loaded erythrocytic cell having a gradient of loaded trehalose (mM) within the erythrocytic cell to extracellular trehalose concentration (mM) within the trehalose solution ranging from about 0.130 to about 0.200. In another embodiment, the loading of the trehalose produces a loaded erythrocytic cell having a gradient of loaded trehalose (mM) within the erythrocytic cell to extracellular trehalose concentration (mM) within the trehalose solution ranging from about 0.04 to about 0.12. In a further embodiment, the loading of the trehalose produces a loaded erythrocytic cell having a gradient of loaded trehalose (mM) within the erythrocytic cell to extracellular trehalose concentration (mM) within the trehalose solution ranging from about 0.04 to about 0.08, or from about 0.08 to about 0.12, depending on the extracellular trehalose concentration and the temperature of the trehalose solution. The trehalose solution may have a trehalose concentration ranging from about 25% to at least about 1000% greater than the intracellular osmolarity of the erythrocytic cell, or at least about 50% greater than the intracellular osmolarity of the erythrocytic cell.

A further embodiment of the invention provides retaining the trehalose in the erythrocytic cell; more specifically washing the erythrocytic cell and retaining the trehalose in the erythrocytic cell during the washing.

The washing of the erythrocytic cell is preferably with a washing buffer, and retention of the trehalose in the erythrocytic cell increases from about 25% to about 175% when a buffer concentration increases from about 50% to about 400%, more preferably from about 50% to about 150% when a buffer concentration increases from about 100% to about 300%, and most preferably from about 75% to about 125% (e.g., about 100%) when a buffer concentration increases from about 150% to about 250% (e.g., about 200%). The washing of the erythrocytic cell with a washing buffer includes employing a ratio of an extracellular buffer concentration (mOsm) to an intracellular trehalose concentration (mM) ranging from about 14.0 to about 4.0, more particularly from about 12.0 to about 5.0, including from about 9.0 to about 6.0 and from about 8.0 to about 7.0 (e.g., about 7.5).

Additional embodiments of the present invention provide a method for loading (e.g., by fluid phase endocytosis) an oligosaccharide into cells (e.g., erythrocytic cells) comprising disposing cells in an oligosaccharide solution having an oligosaccharide concentration of at least about 25% greater than the intracellular osmolarity of the cells for loading oligosaccharide into the cells, and preventing a decrease in a loading gradient in the loading of the oligosaccharide into the cells. In one embodiment of the invention, the preventing a decrease in a loading gradient in the loading of the oligosaccharide into the cells comprises maintaining a concentration of the oligosaccharide in the oligosaccharide solution below a certain concentration, such as below a concentration ranging from about 35 mM to about 65 mM, more particularly below a concentration ranging from about 40 mM to about 60 mM, more particularly further below a concentration ranging from about 45 mM to about 55 mM (e.g., below about 50 mM). In another embodiment the preventing a decrease in a loading gradient in the loading of the oligosaccharide into the cells comprises maintaining a positive gradient of concentration of oligosaccharide loaded into the cells to concentration of the oligosaccharide in the oligosaccharide solution.

Further embodiments of the present invention provide a method for preparing a dehydrated composition comprising loading cells in a loading solution having a salt solution and a solute for producing loaded cells, and lyophilizing the loaded cells in a freeze-drying solution having a drying-salt solution, the solute, an inert substance and a protein to produce a dehydrated composition. The loading solution may comprise at least about 200 mM of the solute and at least about 75 mOsm of the salt solution. The freeze-drying solution may comprise at least about 50 mM of the solute, at least about 2.0% by weight of the inert substance, at least about 0.5% by weight of the protein, and at least about 25 mOsm for an osmolarity of the drying-salt solution.

Embodiments of the present invention additionally provide a method for reconstituting dried cells comprising drying solute-loaded cells in a drying solution having a salt solution (e.g., PBS), a solute (e.g., trehalose), an inert substance (e.g., a starch), and a protein (e.g., albumin) to produce dried cells, and reconstituting the dried cells in a rehydration solution having the salt solution, the solute, the inert substance, and the protein to produce reconstituted cells. The drying solution may comprise at least about 50 mM of the solute, at least about 25 mOsm osmolarity of the salt solution, at least about 2.0% by weight of the inert substance, and at least about 0.5% by weight of the protein. The rehydration solution may comprise at least about 50 mM of the solute, at least about 25 mOsm osmolarity of the salt solution, at least about 2.0% by weight of the inert substance, and at least about 0.5% by weight of the protein. The dried cells may comprise from about 25 mM to about 300 mM of the solute, from about 5 mOsm to about 100 mOsm osmolarity for the salt solution, from about 0.1% by weight to about 2.5% by weight of the protein, and from about 1.0% by weight to about 15.0% by weight of the inert substance. The dried cells may comprise from about 60 mM to about 80 mM trehalose, from about 10 mOsm to about 40 mOsm PBS, from about 0.3% by weight to about 9.0% by weight albumin, and about 1.0% by weight to about 4.0% by weight starch.

Additional embodiments of the invention provide a method for loading a biological sample (e.g., a mammalian biological sample) with a solute comprising disposing a biological sample into a solute solution having a solute and a chemical selected from the group consisting of a monosaccharide (e.g., glucose), a monosaccharide polyol, a cell metabolite-controlling agent, a salt, a buffering salt compound, and mixtures thereof; and incubating the biological sample in the solute solution while maintaining a positive solute concentration increase (mM) to incubation (hours) loading gradient during incubating to load the biological sample with the solute. Incubating the biological sample comprises incubating at a temperature ranging from about 35° C. to about 39° C. and from about 6 hours to about 10 hours. The monosaccharide polyol comprises mannitol, and the cell metabolite-controlling agent comprises adenine.

Additional embodiments of the present invention provide a method for maintaining a viability level of a metabolite in a biological sample comprising disposing a biological sample into a solute solution having a solute and a chemical selected from the group consisting of a monosaccharide, a monosaccharide polyol, a cell metabolite-controlling agent, a salt, and mixtures thereof; and incubating the biological sample in the solute solution while maintaining a viability level of a metabolite in the biological sample. The method may additionally comprise maintaining a positive gradient of metabolite level increase to incubation duration during the incubating. Maintaining a viability level of a metabolite comprises preventing a decrease in a metabolite level of the biological sample. The metabolite may be selected from the group consisting of ATP, 2,3-DPG and mixtures thereof. Incubating the biological sample in the solute solution comprises incubating at a temperature ranging from about 35° C. to about 39° C. for a period of time ranging from about 4 hours to about 8 hours.

Further additional embodiments of the invention provide a method for maintaining a viability level of a metabolite in a biological sample comprising disposing a biological sample into a solute solution comprising a solute and a chemical selected from the group consisting of a monosaccharide, a monosaccharide polyol, a cell metabolite-controlling agent, a salt, and mixtures thereof; and incubating the biological sample in the solute solution while maintaining a maintaining a positive gradient of change in a level of a metabolite (e.g., ATP) to change in duration of incubation.

These provisions, together with the various ancillary provisions and features which will become apparent to those skilled in the art as the following description proceeds, are attained by the processes and cells of the present invention, preferred embodiments thereof being shown with reference to the accompanying drawings, by way of example only, wherein:

### BRIEF DESCRIPTION OF THE DRAWINGS

FIG. 1 graphically illustrates the loading efficiency of trehalose plotted versus incubation temperature of human platelets.

FIG. 2 graphically illustrates the loading efficiency (cytosolic concentration divided by the extracellular concentration, the sum multiplied by 100) following incubation as a function of incubation time;

FIG. 3 graphically illustrates the internal trehalose concentration of human platelets versus external trehalose concentration as a function of temperature at a constant incubation or loading time.

FIG. 4 graphically illustrates the loading efficiency of trehalose into human platelets as a function of external trehalose concentration.

FIG. 5 graphically illustrates intracellular trehalose concentration of human erythrocytes as a function of extracellular trehalose at respective temperatures of 4° C. and 37° C.

FIG. 6 graphically illustrates the fragility index of erythrocytic cells incubated overnight at respective temperatures of 4° C. and 37° C. in the presence of and as a function of increasing intracellular trehalose concentrations.

FIG. 7 graphically illustrates trehalose uptake (i.e., intracellular trehalose mM) and hemolysis (i.e., % hemolysis) as a function of incubation temperature (0° C.).

FIG. 8 graphically illustrates trehalose uptake (i.e., intracellular trehalose mM) as a function of the osmolarity of the washing buffer.

FIG. 9 graphically illustrates % hemolysis of loaded cells vs. time (hours) of incubation of the loaded 300 mOsm PBS buffer.

FIG. 10 graphically illustrates time course (incubation time, hours) of hemolysis(%) of trehalose loaded cells as a function of the trehalose concentration in the incubation buffer.

FIG. 11 graphically illustrates time course (incubation time, hours) of hemolysis(%) of trehalose loaded cells as a function of the composition of the incubation buffer, and illustrating that HES and albumin (HSA) do not have any detrimental effect on cell hemolysis during incubation.

FIG. 12 is a 40× picture of rehydrated erythrocytic cells having no intracellular trehalose prior to freeze-drying.

FIG. 13 is a 40× picture of rehydrated erythrocytic cells having 3 mM intracellular trehalose, after initially trehalose-loading and freeze-drying the cells in 300 mM trehalose/100 mOsm PBS, 15% HES and 2.5% HSA.

FIG. 14 is a 40× picture of rehydrated erythrocytic cells having 60 mM intracellular trehalose, after initially trehalose-loading and freeze-drying the cells in 300 mM trehalose/100 mOsm PBS, 15% HES and 2.5% HSA.

FIG. 15 is a 100× picture of rehydrated erythrocytic cells having 60 mM intracellular trehalose, after initially trehalose-loading and freeze-drying the cells in 300 mM trehalose/100 mOsm PBS, 15% HES and 2.5% HSA.

FIG. 16 is a graph of hemolysis(%) of trehalose loaded, freeze-dried and rehydrated erythrocytic cells as a function of intracellular trehalose concentration (mM), graphically illustrating the effect of cytoplasmic trehalose on the survival of rehydrated erythrocytic cells.

FIG. 17 is a graph of mean corpuscular hemoglobin of trehalose-loaded, freeze-dried and rehydrated erythrocytic cells as a function of intracellular trehalose concentration (mM), graphically illustrating that as the concentration of intracellular trehalose increases for rehydrated erythrocytic cells, the mean corpuscular hemoglobin (the amount of hemoglobin found in intact erythrocytic cells) also increases for rehydrated erythrocytic cells.

FIG. 18 is a graph showing the ATP level of erythrocytes in buffers with different compositions during 5 hours incubation at 38-41° C.

FIG. 19 is a graph showing the level of 2,3-DPG during 5 hours incubation at 38-41° C. in buffers with different composition.

FIG. 20 is a graph showing the effect of pre-hydration time on the survival of freeze-dried and rehydrated erythrocytes.

FIG. 21 is a graph illustrating the results of having studied the effects of α-crystallin on the percent hemolysis.

FIG. 22 is a graph illustrating the combined effect of α-crystallin, Zn²⁺ ions and pre-hydration on the survival of erythrocytic cells.

FIG. 23, is a graph illustrating the effect of rejuvenating buffer on the synthesis of ATP and 2,3-DPG in rehydrated erythrocytes.

FIG. 24 is a graph of trehalose uptake (e.g., cytoplasmic trehalose concentration mM) as a function of time of incubation in a solute solution comprising 800 mM trehalose, 100 mOsm ADSOL, and 6.6 mM K-phosphate (pH 7.2).

FIG. 25 is a graph of percent hemolysis of erythrocytes (RBCs) at 4° C., 23° C., 37° C. and 41° C. as a function of time of incubation in a solute solution comprising 800 mM trehalose, 100 mOsm ADSOL, and 6.6 K-phosphate (pH 7.2).

FIG. 26 is a graph of ATP (μmol/g Hb) levels (or quantities) for control RBCs and for trehalose-loaded RBCs during incubation in 800 mM trehalose/100 mOsm ADSOL/6.6 mM K-phosphate at 4° and 37° C.

FIG. 27 is a graph of 2,3-DPG (μmol/g Hb) levels (or quantities) for control RBCs and for trehalose-loaded RBCs during incubation in 800 mM trehalose/100 mOsm ADSOL/6.6 mM K-phosphate at 4° and 37° C.

### DETAILED DESCRIPTION OF PREFERRED EMBODIMENTS OF THE INVENTION

Compositions and embodiments of the invention include methods for loading solutes into cells, as well as cells that have been manipulated (e.g., by freeze-drying) or modified (e.g., loaded with a chemical or drug) in accordance with methods of the present invention. The cells may be any type of cell including, not by way of limitation, erythrocytic cells, eukaryotic cells or any other cell, whether nucleated or non-nucleated.

The term “erythrocytic cell” is used to mean any red blood cell. Mammalian, particularly human, erythrocytes are preferred. Suitable mammalian species for providing erythrocytic cells include by way of example only, not only human, but also equine, canine, feline, or endangered species.

The term “eukaryotic cell” is used to mean any nucleated cell, i.e., a cell that possesses a nucleus surrounded by a nuclear membrane, as well as any cell that is derived by terminal differentiation from a nucleated cell, even though the derived cell is not nucleated. Examples of the latter are terminally differentiated human red blood cells. Mammalian, and particularly human, eukaryotes are preferred. Suitable mammalian species include by way of example only not only human, but also equine, canine, feline, or endangered species.

Molarity, or millimolarity, mM, is the number of moles (or millimoles) of a solute per liter of solution and is a measure of the concentration. Osmolarity (Osm), or milliosmolarity (mOsm), is a count of the number of dissolved particles per liter of solution and is a measure of the osmotic pressure exerted by solutes. Biological membranes, such as cell membranes, can be semi-permeable because they allow water and some small molecules to pass, but block the passage of proteins or macromolecules. Since the osmolarity of a solution is equal to the molarity times the number of particles per molecule, 600 mM trehalose is equal to 600 mOsm trehalose because trehalose does not dissociate in water. However, with respect to compounds that dissociate in water, such as NaCl, 1 mM NaCl is equal to 2 mOsm NaCl because it has two particles. Similarly, 100 mM NaCl is equal to 200 mOsm NaCl. Thus, for a 300 mOsm PBS buffer (100 mM NaCl, 9.4 mM Na₂HPO₄, 0.6 mm KH₂PO₄, pH 7.4), 300 mOsm refers to all of the osmotically active particles in the PBS solution, with 200 mOsm of the 300 mOsm stemming from NaCl.

Broadly, the preparation of solute-loaded cells in accordance with embodiments of the invention comprises the steps of loading one or more cells with a solute by placing one or more cells in a solute solution having a solute concentration of sufficient magnitude to produce hyperosmotic pressure on the cell for transferring the solute from the solution into the cell. For increasing the transfer or uptake of the solute from the solute solution, the solute solution temperature or incubation temperature has a temperature above about 25° C., more preferably above 30° C., such as from about 30° C. to about 40° C. In another embodiment of the invention, a solute solution (e.g., trehalose solution) has a solute (e.g., trehalose) concentration of at least about 25%, preferably at least about 50%, greater than the intracellular osmolarity of the cells for loading the solute into the cells.

For various embodiments of the invention, a solute solution has a solute concentration ranging from about 25% to at least about 1000% greater than the intracellular osmolarity of the cell. For additional various embodiments of the invention, the solute solution, especially when the solute solution is employed as a loading buffer, has a solute concentration ranging from about 320 mM to bout 4000 mM, preferably from about 320 mM to about 2000 mM, more preferably from about 500 mM to about 1000 mM.

The method(s) for various embodiments of the present invention may additionally comprise preventing a decrease in a loading gradient and/or a loading efficiency gradient in the loading of the solute into the cells. Preventing a decrease in a loading efficiency gradient in the loading of the solute into the cells comprises maintaining a positive gradient of loading efficiency (e.g., in %) to concentration (e.g., in mM) of the solute in the solute solution. Preventing a decrease in a loading gradient in the loading of the solute into the cells comprises maintaining a concentration of the solute in the solute solution below a certain concentration (e.g., below a concentration ranging from about 35 mM to about 65 mM, more particularly below from about 40 mM to about 60 mM, or below from about 45 mM to about 55 mM, such as below about 50 mM); and/or maintaining a positive gradient of concentration of solute loaded into the cells to concentration of the solute in the solute solution.

The solute solution for various embodiments of the present invention may be used for loading and/or washing and/or freeze-drying and/or rehydration, or for any other suitable purpose. When the solute solution is employed for loading a solute into the cells, the solute solution may be any suitable physiologically acceptable solution in an amount and under conditions effective to cause uptake or “introduction” of the solute from the solute solution into the cells. A physiologically acceptable solution is a suitable solute-loading buffer, such as any of the buffers stated in the previously mentioned related patent applications, all having been incorporated herein by reference thereto. The solute solution may also be any suitable physiologically acceptable solution in an amount and under conditions effective for washing and/or freeze drying and/or rehydration. Therefore, the solute solution may be used as a washing buffer for washing loaded cells and/or as a freeze-drying buffer for freeze-drying loaded cells and/or as a rehydration buffer for rehydrating thawed cells in reconstituting cells. Thus, any of the solute solutions for embodiments of the present invention may be used for any suitable purpose, including loading, washing, freeze-drying, and rehydration.

For particular embodiments of the present invention, especially when the solute solution is being employed as a loading buffer and/or washing buffer, the solute solution comprises a solute and a salt solution. In other particular embodiments of the invention, especially when the solute solution is being employed as a freeze-drying buffer and/or a rehydration buffer, the solute solution comprises a salt solution (e.g., PBS), a protein, a solute, and at least one inert substance. However, it is to be understood that the solute solution comprising a salt solution, a protein, a solute, and an inert substance may be used for any other suitable purpose including for loading a solute into cells and for washing solute-loaded cells.

Protein, when referred to herein, means any suitable protein (e.g., simple or conjugated protein), including any complex, high polymer containing carbon, hydrogen, oxygen, nitrogen, and usually sulfur, and composed of chains of amino acids connected by peptide linkages. Protein includes albumin, which when referred to herein means any suitable albumin (e.g., bovine albumin), including any of a group of water-soluble proteins of wide occurrence in such natural products as milk (lactalbumin), blood serum, eggs (ovalbumin). Preferably, the albumin comprises human serum albumin (HSA).

The solute is preferably a carbohydrate (e.g., an oligosaccharide) selected from the following groups of carbohydrates: a monosaccharide (e.g., bioses, trioses, tetroses, pentoses, hexoses, heptoses, etc), a disaccharide (e.g., lactose, maltose, sucrose, melibiose, trehalose, etc), a trisaccharide (e.g., raffinose, melezitose, etc), or tetrasaccharides (e.g., lupeose, stachyose, etc), and a polysaccharide (e.g., dextrins, starch groups, cellulose groups, etc). More preferably, the solute is a disaccharide, with trehalose being the preferred, particularly since it has been discovered that trehalose does not degrade or reduce in complexity upon being loaded. Thus, in the practice of various embodiments of the invention, trehalose is transferred from a solution into the cells without degradation of the trehalose.

The salt solution may be any suitable physiologically acceptable solution in an amount and under conditions effective to function as a carrier medium for a solvent, or for a mixture of a solvent, a protein and/or an inert substance. The salt solution may comprise a phosphate buffered saline (PBS) solution comprising NaCl, Na₂HPO₄, and KH₂PO₄. A suitable PBS buffer is 100 mOsm PBS buffer (51.3 mM NaCl, 1.87 mM Na₂HPO₄, 0.35 mM KH₂PO₄, pH 7.2).

The inert substance is preferably a carbohydrate, such as any of the carbohydrates previously mentioned above. Preferably, the inert substance comprises a polysaccharide. More preferably, the inert substance comprises a starch, such as, by way of example only, hydroxy ethyl starch (HES).

The quantities of solute, protein and inert substance employed in the solute solution, more specifically in combination with a saline solution, are of suitable quantities and proportion for minimizing the loss or destruction of cells, more particularly for minimizing hemolysis, especially after freeze-drying and reconstitution (e.g., prehydration and rehydration), and/or especially when the solute solution is employed as a freeze-drying buffer and/or rehydration buffer.

For various embodiments of the present invention, the solute solution comprises: a solute and a salt solution. The concentration of the solute in the solute solution may be at least about 50 mM, such as ranging from about 50 mM to about 3000 mM, preferably from about 100 mM to about 1500 mM, more preferably from about 150 mM to about 1000 mM, most preferably from about 200 mM to about 600 mM. The osmolarity of the salt solution may be at least about 25 mOsm, such as ranging from about 25 mOsm to about 1000 mOsm, preferably from about 50 mOsm to about 300 mOsm, more preferably from about 75 mOsm to about 200 mOsm. The solute solution comprising a solute and a salt solution may be used for any suitable purpose including as a loading buffer and/or as a washing buffer.

For additional various embodiments of the present invention, the solute solution may further comprise (in addition to the solute and the salt solution) a protein and/or an inert substance. The amount or quantity of the inert substance (e.g., HES) in the solute solution may be at least about 2.0% by weight, such as ranging from about 2.0% by weight to about 50% by weight, preferably from about 5% by weight to about 35% by weight, more preferably from about 10% by weight to about 30% by weight, most preferably from about 12% by weight to about 20% by weight (e.g., about 15% by weight). The amount or quantity of the protein (e.g. HSA) in the solute solution may be at least about 0.5% by weight, such as ranging from about 0.5% by weight to about 15% by weight, preferably from about 1% by weight to about 10% by weight, more preferably from about 1.5% by weight to about 8% by weight, most preferably from about 1.5% by weight to about 5% by weight (e.g., about 2.5% by weight). The solute solution comprising a solute, a salt solution, a protein and/or an inert substance may be used for any suitable purpose including as a freeze drying buffer and/or rehydration buffer.

An extracellular medium of about 280-320 mOsm is considered iso-osmotic for cells, particularly erythrocytic cells, with regard to the amount of permeable solutes in the cytoplasm. Any increase of the amount of solutes in the intracellular medium creates an osmotic shock, ranging from a mild shock at about 350 mM trehalose to a strong shock at about 420 mM trehalose, and a leakage of water which would reversibly reduce the cell volume. However, small molecular weight solutes, such as trehalose, in an extracellular medium in a concentration higher than about 320 mM, can pass through the membrane of a cell using a diffusion vector. It has been discovered that an extracellular concentration of trehalose higher than about 450 mM (or mOsm), which is about 50% greater than an intracellular milliosmolarity, will produce an osmotic shock that will result in trehalose uptake. Increasing the extracellular trehalose concentration leads to even higher osmotic shock and higher trehalose uptake.

Other embodiments of the present invention provide for retaining a solute in a cell. Preferably, after the cells have been loaded with a solute, such as an oligosaccharide (e.g., trehalose), the cells are then washed. More preferably, during the washing of the cells the solute is retained in the cells. Washing leads to hemolysis of the fragile cells and removal of cellular fragments and free hemoglobin. The net result is that the remaining cells do indeed have an elevated trehalose content. The washing may be with a washing solution (e.g., such as a washing buffer having an oligosaccharide), and retention of the solute in the cell increases from about 25% to about 175% when a buffer concentration (e.g., the osmolarity of all osmotically active particles within the washing buffer solution) increases from about 50% to about 400%, more preferably from about 50% to about 150% when a buffer concentration increases from about 100% to about 300%, and most preferably from about 75% to about 125% (e.g., about 100%) when a buffer concentration increases from about 150% to about 250% (e.g., about 200%). The washing of the cell with a washing buffer includes employing a ratio of a buffer concentration (mOsm) (e.g., an extracellular buffer concentration) to an intracellular solute concentration (mM) ranging from about 14.0 to about 4.0, such as from about 12.0 to about 5.0, including from about 9.0 to about 6.0 and from about 8.0 to about 7.0 (e.g., about 7.5).

As indicated in patent application Ser. No. 10/052,162, which claims the benefit of patent application Ser. No. 09/501,773, filed Feb. 10, 2000, with respect to common subject matter, the amount of the preferred trehalose loaded inside the cells ranges from about 10 mM to about 50 mM, and is achieved by incubating the cells to preserve biological properties during freeze-drying with a trehalose solution, preferably a trehalose solution that has up to about 50 mM trehalose therein. Higher concentrations of trehalose during incubation are not preferred, particularly since an embodiment of the invention includes preventing a decrease in a loading gradient, or a loading efficiency gradient, in the loading of the solute into the cell. It has been discovered that preventing a decrease in a loading gradient, or a loading efficiency gradient, in the loading of a solute (e.g., an oligosaccharide, such as trehalose) into a cell comprises maintaining a concentration of the solute in the solute solution below a certain concentration (e.g., below about 75 mM, such as below about a concentration ranging from about 35 mM to about 65 mM, more particularly below from about 40 mM to about 60 mM, or below from about 45 mM to about 55 mM, such as below about 50 mM). It has been further discovered that preventing a decrease in a loading gradient, or a loading efficiency gradient, in the loading of a solute into a cell comprises maintaining a positive gradient of loading efficiency to concentration of the solute in the solute solution.

As further indicated in co-pending patent application Ser. No. 10/052,162, the effective loading of trehalose is also accomplished by means of using an elevated temperature of from greater than about 25° C. to less than about 40° C., more preferably from about 30° C. to less than about 40° C., most preferably about 37° C. This is due to the discovery of the second phase transition for cells.

Referring now to FIG. 1, there is seen a graphical illustration from co-pending patent application Ser. No. 10/052,162 of the loading efficiency of trehalose plotted versus incubation temperature of human platelets. The trehalose loading efficiency begins a steep slope increase at incubation temperatures above about 25° C. and continues up to about 40° C. The trehalose concentration in the exterior solution (that is, the solute solution or loading buffer) and the temperature during incubation together lead to a trehalose uptake that occurs through fluid phase endocytosis. Example 1 below provides the more specific testing conditions and parameters which produced the graphical illustrations of FIG. 1. It is believed that the graphical illustration of the loading efficiency in FIG. 1 could be generally applicable for cells in general.

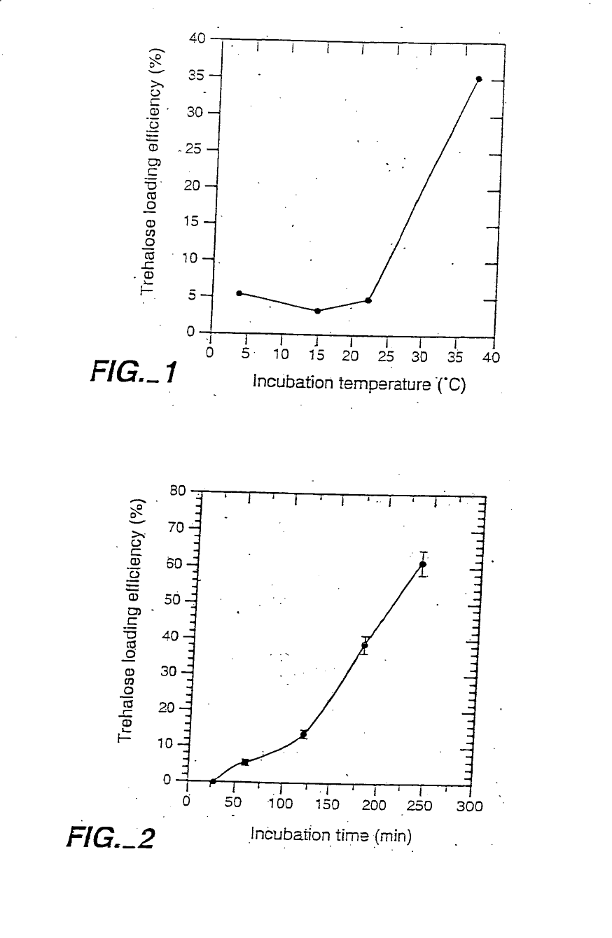

Referring now to FIG. 2, there is seen an illustration from co-pending patent application Ser. No. 10/052,162 of trehalose loading efficiency for human blood platelets as a function of incubation time. More specifically, FIG. 2 is a graphical illustration of the loading efficiency (cytosolic concentration divided by the extracellular concentration, the sum multiplied by 100) following incubation as a function of incubation time. Example 1 below provides the more specific testing conditions and parameters which produced the graphical illustrations of FIG. 2. It is believed that the graphical illustration of the loading efficiency in FIG. 2 could also be generally applicable for cells in general.

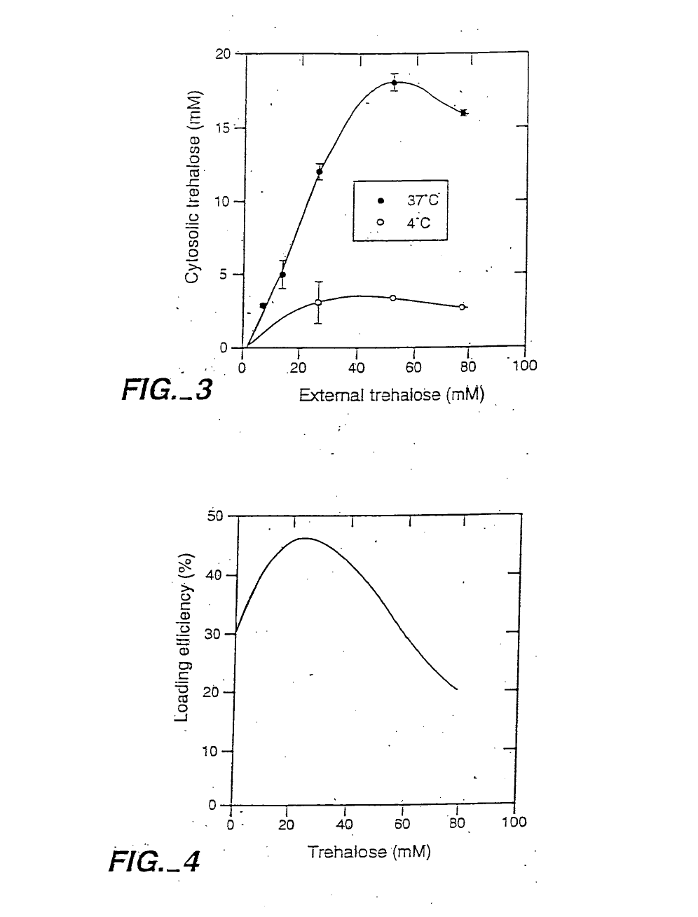

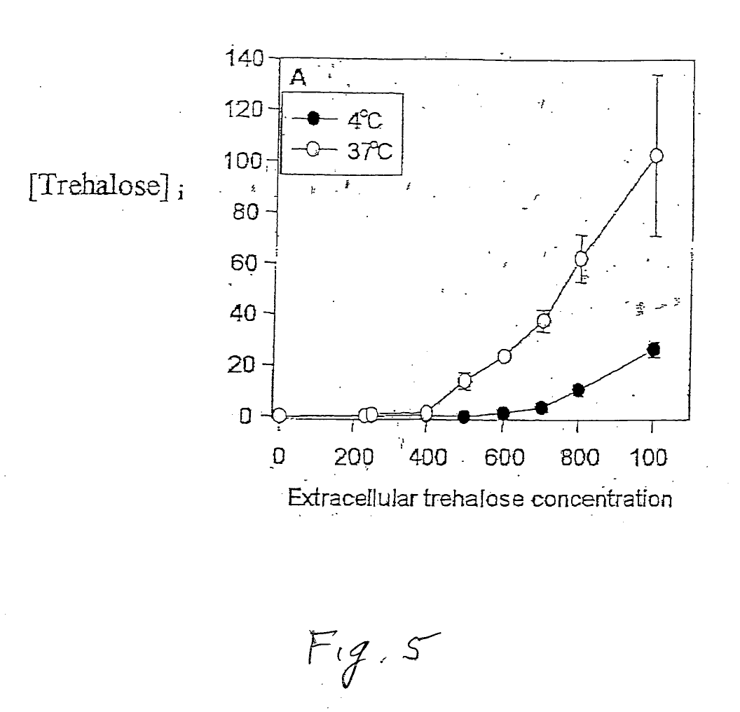

Referring now to FIG. 3, there is seen a graphical illustration from patent application Ser. No. 10/052,162 of the internal trehalose concentration of human platelets versus external trehalose concentration as a function of 4° C. and 37° C. temperatures at a constant incubation or loading time. In FIG. 4 there is seen a graphical illustration from patent application Ser. No. 10/052,162 of the loading efficiency of trehalose into human platelets as a function of external trehalose concentration. Example 1 below provides the more specific testing conditions and parameters which produced the graphical illustrations of FIGS. 3 and 4. In additional embodiments of the present invention, it is further believed that the general-findings illustrated in FIGS. 3 and 4 with respect to platelets are generally broadly applicable to cells in general.

Thus, applying the findings illustrated in FIG. 3 and in FIG. 4 to solutes and cells in general, a decrease in a loading gradient or a loading efficiency gradient in the loading of a solute into a cell may be prevented. For an embodiment of the present invention and as broadly illustrated in FIG. 3, preventing a decrease in a loading gradient or a loading efficiency gradient in the loading of the solute (e.g., an oligosaccharide such as trehalose) into the cell comprises maintaining a concentration of the solute (e.g., an oligosaccharide such as trehalose) in the solute solution (e.g. an oligosaccharide solution such as a trehalose solution) below a solute concentration ranging from about 35 mM to about 65 mM, more specifically a solute concentration ranging from about 40 mM to about 60 mM, more specifically further a solute concentration ranging from about 45 mM to about 55 mM (e.g., about 50 mM). In another embodiment of the present invention and as best illustrated in FIG. 4, preventing a decrease in a loading gradient or a loading efficiency gradient in the loading of the solute (e.g., an oligosaccharide, such as trehalose) into the cell comprises maintaining a positive gradient of loading efficiency (e.g., loading efficiency in %) to concentration (e.g., concentration in mM) of the solute in the solute solution (e.g. an oligosaccharide solution, such as a trehalose solution).

When a solute is loaded from a solute solution into one or more cells, the solute solution preferably has a solute concentration of sufficient magnitude to produce hyperosmotic pressure on the one or more cells. It has been discovered that the basis for the loading of the solute into the cells is dependent upon osmotic shock. The magnitude of osmotic shock and hyperosmotic pressure on the cells depends on the difference between internal solute concentration, or the intracellular osmolarity, within the cells, and the external solute concentration within the solute solution, or the extracellular cellular solute concentration. For embodiments of the invention, the solute solution has a solute concentration ranging from about 320 mM to about 4000 mM, preferably from about 320 mM to about 2000 mM, more preferably from about 500 mM to about 1000 mM.

In another embodiment of the present invention, the solute solution, especially when used for loading the solute into one or more cells, may comprise a solute and a salt solution having a suitable osmolarity (mOsm). Preferably, the ratio of the osmolarity (mOsm) of the salt solution to the solute concentration (mM) in the solution ranges from about 0.04 to about 1.0, preferably from about 0.05 to about 0.50, more preferably from about 0.07 to about 0.30, most preferably from about 0.10 to about 0.20. The osmolarity (mOsm) of the solute solution for these embodiments of the invention is the osmolarity of the osmotically active particles except, or other than, the osmolarity of the solute. As indicated previously, the osmolarity of the solute solution may range from about 25 mOsm to about 1000 mOsm, preferably from about 50 mOsm to about 300 mOsm, more preferably from about 75 mOsm to about 200 mOsm. As indicated previously, the solute solution may be any suitable solution for purposes for embodiments of the present invention. Preferably, the solute solution comprises a salt solution, such as a phosphate buffered saline (PBS) comprising NaCl, Na₂HPO₄, and KH₂PO₄. A suitable PBS buffer is 100 mOsm PBS buffer (51.3 mM NaCl, 1.87 mM Na₂HPO₄, 0.35 mM KH₂PO₄, pH 7.2).

It has also been discovered that the basis for the loading of the solute into the cells is not only dependent upon osmotic shock, but is also dependent upon the thermal effects on flux of the solute across the membranes of the cells. The higher the thermal effects on flux of the solute across the membranes of the cells, the larger the amount of solute loaded into the cells. Stated alternatively, loading of a solute into cells increases as the temperature of the solute solution increases. Referring now to FIG. 5, there is seen a graphical illustration of intracellular trehalose concentration as a function of extracellular trehalose at respective temperatures of 4° C. and 37° C. Thus, at a temperature ranging from about 30° C. to about 4° C. (e.g. at about 37° C.) a gradient of a solute concentration (M), such as an oligosaccharide (e.g., trehalose) concentration, within a cell (e.g., an erythrocytic cell) to extracellular solute concentration (M) within a loading solution (or buffer) ranges from about 0.130 to about 0.200. At a temperature ranging from about 0° C. to about 10° C. (e.g. at about 4° C.) a gradient of a solute concentration (M), such as an oligosaccharide (e.g., trehalose) concentration, within a cell (e.g., an erythrocytic cell) to extracellular solute concentration (M) within a loading solution (or buffer) ranges from about 0.04 to about 0.12, more specifically from about 0.04 to about 0.08, and from about 0.08 to about 0.12, depending on the quantity of extracellular solute concentration. Example 2 below provides the more specific testing conditions and parameters which produced the graphical illustrations of FIG. 5.

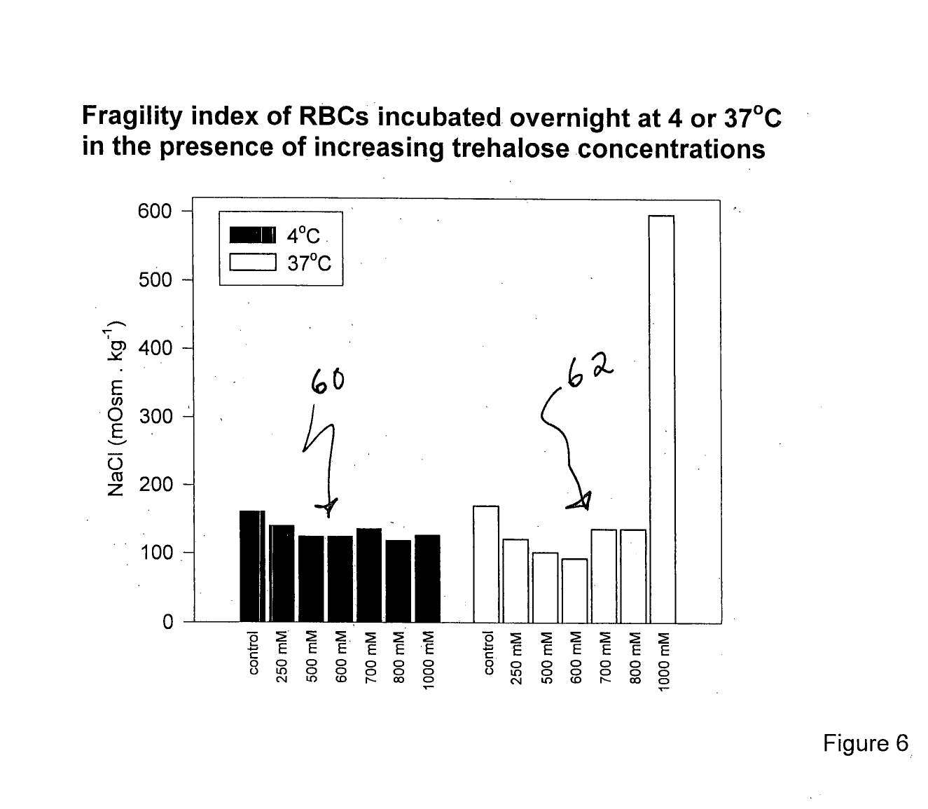

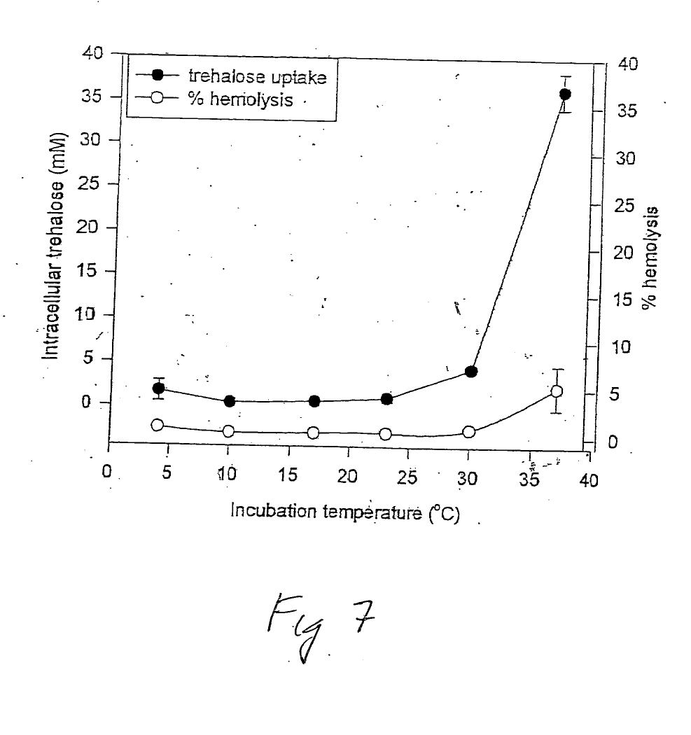

Referring now to FIG. 6, there is seen a graphical illustration of the fragility index of erythrocytic cells incubated overnight at respective temperatures of 4° C. and 37° C. in the presence of and as a function of increasing intracellular trehalose concentrations. The osmotic fragility index was generated by the extent of hemolysis as a function of the NaCl concentration. The graphical illustration of FIG. 6 represents a test for investigating the effects of hyperosmotic treatment rendering erythrocytic cells more sensitive to change in intracellular osmolarity. NaCl was loaded into erythrocytic cells from a 100 mOsm PBS buffer at loading 100 mOsm PBS buffer temperatures of 4° C. and 37° C. for extracellular trehalose concentrations of 0 mM (control cells), 250 mM, 500 mM, 600 mM, 700 mM, 800 mM and 1000 mM. Data blocks, respectively generally indicated as 60 and 62, represent the intracellular trehalose concentrations for 100 mOsm PBS solution loading temperatures of 4° C. and 37° C. The mOsm/kg values of NaCl represent extracellular NaCl osmolarity of the erythrocytic cells resulting from the transfer of NaCl from the PBS loading buffer into the erythrocytic cells. The erythrocytic cells that had been loaded in trehalose solutions (between 250 mM and 1000 mM) in 100 mOsm PBS were suspended in increasing concentrations of NaCl (between 50 and 600 mOsm NaCl). The percent hemolysis measured after resuspending the loaded cells in NaCl represents the fragility index. The data show that the erythrocytic cells were stable osmotically in trehalose media with concentrations between 250 mM and 800 mM trehalose at both 37° C. and 4° C. In 1000 mM trehalose at 37° C., there is a high increase in the fragility index suggesting that the cells were unstable in this medium (1000 mM trehalose in 100 mOsm PBS). Clearly, at moderate intracellular concentrations of trehalose, osmotic fragility as measured by a standard assay was not severely altered. Thus, erythrocytic cells may be loaded with trehalose concentrations up to about 900 mM (i.e., a trehalose concentration between 800 mM and 1000 mM). Example 3 below provides specific testing conditions and parameters which produced the graphical illustrations of FIG. 6.

Thus, from the findings graphically illustrated in FIGS. 5 and 6, and as more fully explained in Examples 2 and 3 below, temperature of a solute loading solution has an effect in loading a solute from a solute solution into a cell. The effects of temperature, as well as cellular hemolysis, of a trehalose loading solution in loading of trehalose into a cell was tested. The test results are illustrated in FIG. 7, which is a graphical illustration of trehalose uptake (i.e., intracellular trehalose mM) and hemolysis (i.e., % hemolysis) as a function of incubation temperature (° C.). The incubation time was about 6 hours and the medium contained about 800 mM trehalose/100 mM PBS. FIG. 7 illustrates that effective loading occurs above 30° C., and that as the loading temperature of the trehalose loading solution increases, there is slight hemolysis. Example 4 below provides the more specific testing conditions and parameters which produced the graphical illustrations of FIG. 7.

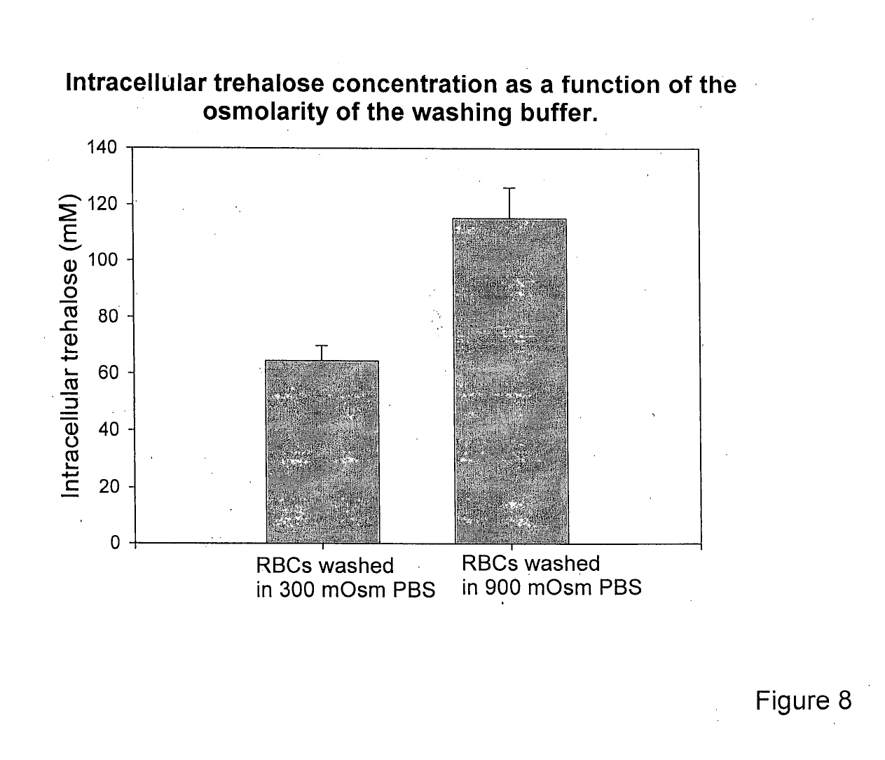

As previously indicated, after a cell (e.g., an erythrocytic cell) has been loaded with a solute (e.g., trehalose), further embodiments of the present invention provide for retaining the solute in the cells. One means for retaining solute within solute-loaded cells is to wash the cells, more specifically by washing the cells and retaining the solute in the cells during the washing. As also previously indicated, the washing of the cells is preferably with a washing buffer. It has been discovered that retention of the solute in the cells increases from about 25% to about 175% when a buffer concentration (e.g., the osmolarity of all osmotically active particles within the washing buffer solution) increases from about 50% to about 400%, more preferably from about 50% to about 150% when a buffer concentration increases from about 100% to about 300%, and most preferably from about 75% to about 125% (e.g., about 100%) when a buffer concentration increases from about 150% to about 250% (e.g., about 200%). It has been further discovered that the washing of the cells with a washing buffer includes employing a ratio of an extracellular buffer concentration (mOsm) to an intracellular trehalose concentration (mM) ranging from about 14.0 to about 4.0, more particularly from about 12.0 to about 5.0, including from about 9.0 to about 6.0 and from about 8.0 to about 7.0 (e.g., about 7.5). Thus, because solute loaded cells are hyperosmotic to a washing buffer, increasing the extracellular osmolarity increases retention of the solute, particularly during washing of the cells, as shown in FIG. 8 which graphically illustrates intracellular trehalose concentration (mM) as a function of the osmolarity of the washing buffer. As shown in FIG. 8, when the extracellular buffer concentration was increased from 300 mOsm PBS to 900 mOsm PBS during washing, the final intracellular trehalose concentration doubled. The 300 mOsm PBS had no trehalose concentration, and the 900 mOsm PBS also had no trehalose concentration. Example 5 below provides the more specific testing conditions and parameters which produced the graphical illustrations of FIG. 8.

After the cells have been effectively loaded with a solute and subsequently washed, the cells may then be contacted with a drying buffer. The drying buffer should include the solute, preferably in amounts up to about 100 mM. The solute in the drying buffer assists in spatially separating the cells as well as stabilizing the cell membranes on the exterior. The drying buffer preferably also includes a bulking agent (to further separate the cells s). Albumin may serve as a bulking agent, but other polymers may be used with the same effect. If albumin is used, it is preferably from the same species as the cells. Suitable other polymers, for example, are water-soluble polymers such as HES (hydroxy ethyl starch) and dextran.

For other embodiments of the present invention, and as previously mentioned, the solute solution may serve as the drying buffer. The solute solution, when functioning as a drying buffer, may comprise at least about 50 mM of the solute, at least about 2.0% by weight of the inert substance, at least about 0.5% by weight of the protein, and at least about 25 mOsm for an osmolarity of the salt solution. More specifically, as indicated, the solute solution for drying buffer purposes may comprise the solute having a concentration ranging from about 50 mM to about 3000 mM, preferably from about 100 mM to about 1500 mM, more preferably from about 150 mM to about 1000 mM, most preferably from about 200 mM to about 600 mM. The osmolarity of the salt solution in the solute solution may range from about 25 mOsm to about 1000 mOsm, preferably from about 50 mOsm to about 300 mOsm, more preferably from about 75 mOsm to about 200 mOsm. The amount or quantity of the inert substance (e.g., HES) in the solute solution may range from about 2.0% by weight to about 50% by weight, preferably from about 5% by weight to about 35% by weight, more preferably from about 10% by weight to about 30% by weight, most preferably from about 12% by weight to about 20% by weight (e.g., about 15% by weight). The amount or quantity of the protein (e.g. HSA) in the solute solution may range from about 0.5% by weight to about 15% by weight, preferably from about 1% by weight to about 10% by weight, more preferably from about 1.5% by weight to about 8% by weight, most preferably from about 1.5% by weight to about 5% by weight (e.g., about 2.5% by weight).

The solute loaded cells in the drying buffer may then be dried while simultaneously cooled to a temperature below about −32° C. A cooling, that is, freezing, rate is preferably between −30° C. and −1° C./min. and more preferably between about −2° C./min to −5° C./min. Drying may be continued until about 95 weight percent of water has been removed from the cells. During the initial stages of lyophilization, the pressure is preferably at about 10×10⁻⁶ torr. As the samples dry, the temperature can be raised to be warmer than −32° C. Based upon the bulk of the sample, the temperature and the pressure it can be empirically determined what the most efficient temperature values should be in order to maximize the evaporative water loss. Freeze-dried cell compositions preferably have less than about 5 weight percent water.

After freeze drying and storage of the cells, the process of using such a dehydrated cell composition comprises rehydrating the cells. The rehydration preferably includes a prehydration step, sufficient to bring the water content of the freeze-dried cells to between about 20 weight percent and about 50 percent, preferably from about 20 weight percent to about 40 weight percent. More preferably, when reconstitution of the freeze dried cells is desired, the freeze dried cells are prehydrated in moisture saturated air at about 37° C. for about one hour to about three hours, followed by rehydration. Use of prehydration yields cells with a much more dense appearance and with no balloon cells being present. The preferred prehydration step brings the water content of the freeze-dried cells to between about 20 weight percent to about 50 weight percent. Rehydration or the prehydrated cells may be with any aqueous based solutions, depending upon the intended application.

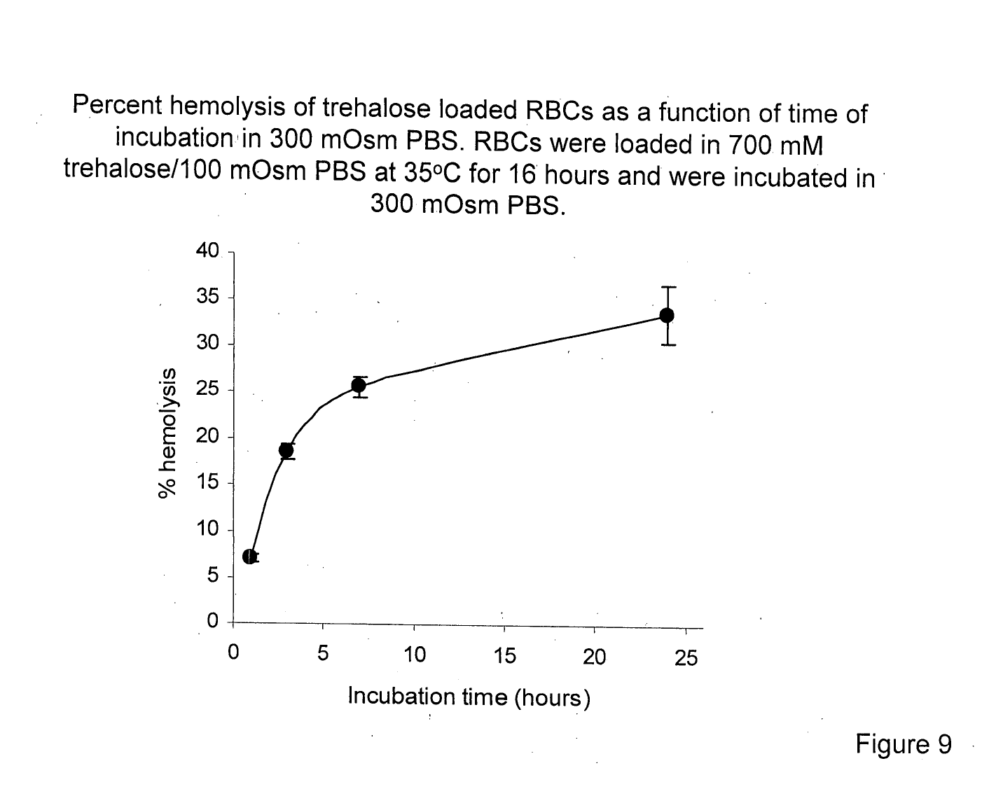

Referring now to FIG. 9, there is seen a graphical illustration of hemolysis (%) of loaded cells as a function of the time (in hours) in 300 mOsm PBS. The cells were loaded with 700 mM trehalose in 100 mM PBS. As illustrated in FIG. 9, the % hemolysis of loaded cells is below about 10% (i.e., about 7%) if the loaded cells are stored in the 300 mOsm PBS within or less than about 3 hours (e.g., within or less than about 2 hours) after being loaded, preferably after being loaded and subsequently washed. The % hemolysis of the loaded cells precipitously increases (i.e., greater than about 10% hemolysis) if more than 3 hours lapses after loading and/or washing loaded cells and incubating them in 300 mOsm PBS. Example 6 below provides the more specific testing conditions and parameters which produced the graphical illustrations of FIG. 9.

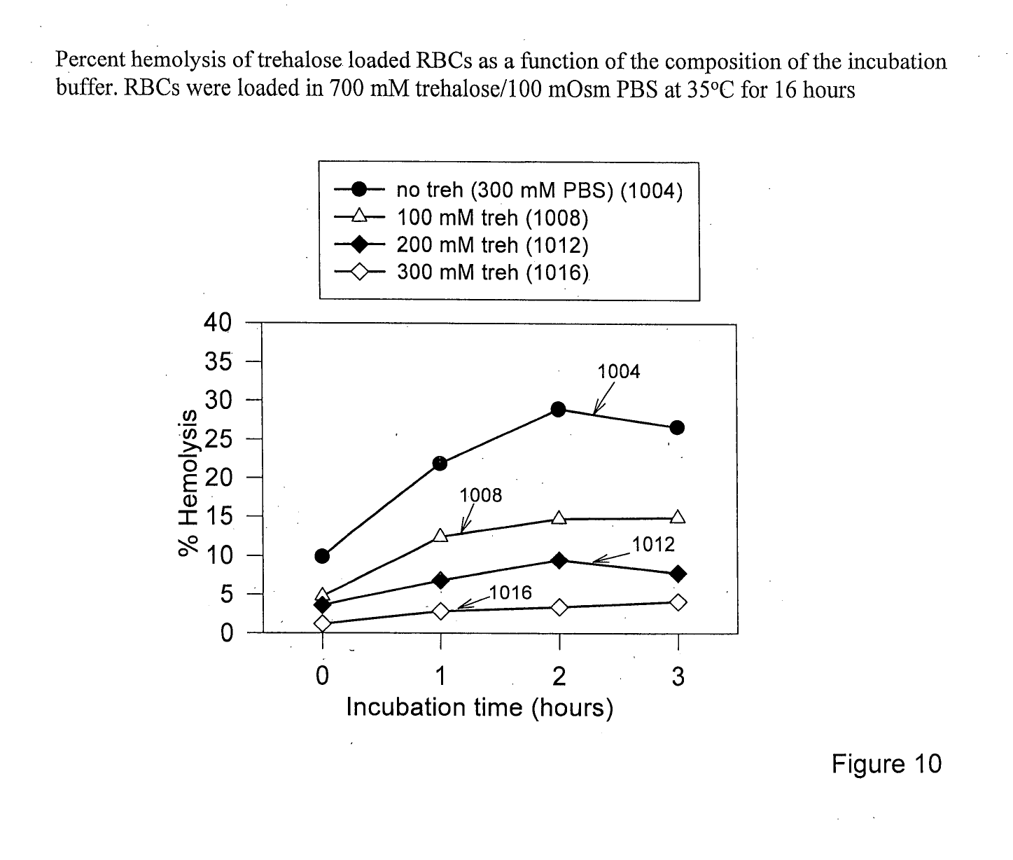

Hemolysis of the cells not only depends on the period of time of subsequently freeze-drying after loading the cells, or after washing loaded cells, but also on the incubation time and the quantity of intracellular solute loaded in the cells, as well as, in some cases, on whether or not the inert substance and/or protein has been admixed with the solute in the salt solution, and/or, in other cases, the quantity of the inert substance and/or the protein that has been added. Referring now to FIG. 10, there is seen a graphical illustration of time course (incubation time, hours) of hemolysis(%) of trehalose-loaded cells as a function of the composition of the incubation buffer. Percent (%) hemolysis represents the hemolysis following incubation in a designated incubation buffer, which typically, may be different from a loading buffer. FIG. 10 represents an illustration of the % hemolysis of trehalose-loaded cells in buffers with different concentration of trehalose. Broadly, as the concentration of the solute (e.g., trehalose) increases, hemolysis decreases. Curve 1004 represents % hemolysis vs. incubation time (hours) when incubating cells with no trehalose and a 300 mM PBS salt solution. Curve 1008 represents % hemolysis vs. incubation time (hours) when incubating cells with 100 mM_trehalose and a 300 mM PBS salt solution. Curve 1012 represents % hemolysis vs. incubation time (hours) when incubating cells with 200 mM trehalose and a 300 mM PBS salt solution. Curve 1016 represents % hemolysis vs. incubation time (hours) when incubating cells with 300 mM_trehalose and a 300 mM PBS salt solution. Thus, broadly, when trehalose-loaded cells are incubated in an incubation period (e.g. from 0 hours to about 3 hours) with a solution comprising a salt solution (e.g., 300 mM PBS) and at least about 200 mM of a solute (e.g., trehalose), hemolysis of the cells is reduced to less than about 10%, more preferably to less than about 5%. Example 7 below provides the more specific testing conditions and parameters which produced the graphical illustrations of FIG. 10.

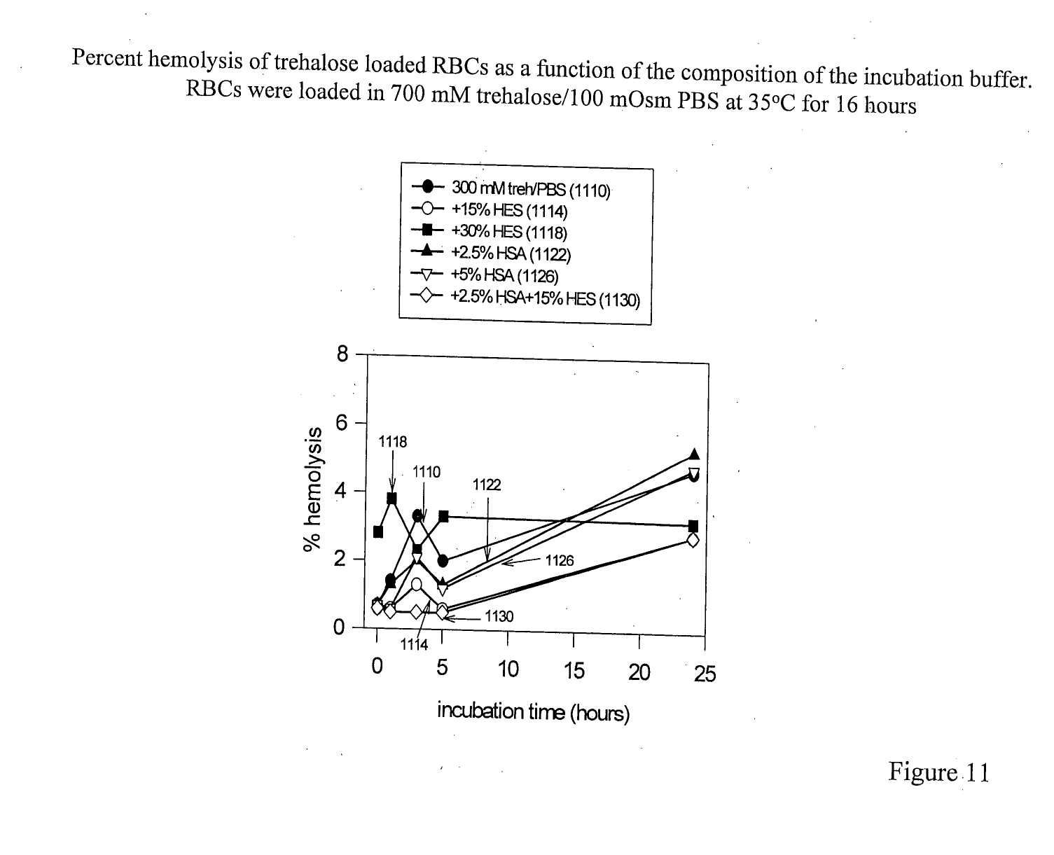

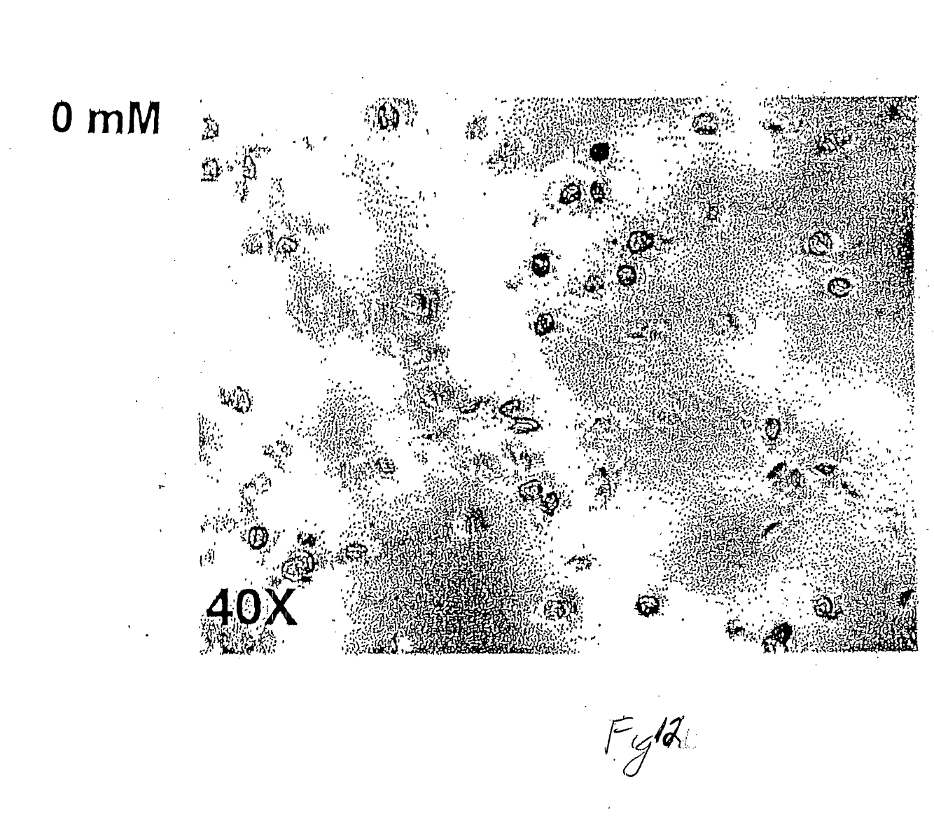

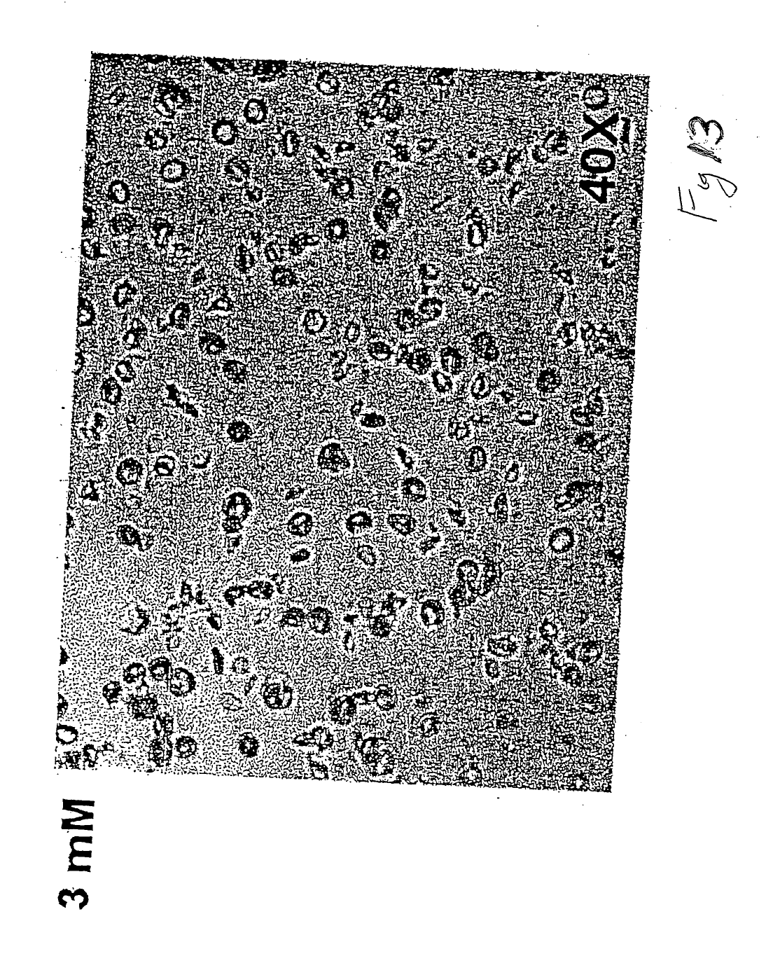

Referring now to FIG. 11, there is seen a graphical illustration of the time course (incubation time, hours) of hemolysis(%) of trehalose-loaded cells as a function of the composition of the incubation buffer, and illustrating that HES and albumin (HSA) do not have any detrimental effect on cell hemolysis during incubation. FIG. 11 illustrates % hemolysis of trehalose-loaded cells in buffers with different amount of HES and HSA for determining which buffers provide highest cell stability, assessed as the lowest % hemolysis. As indicated, hemolysis of the cells not only depends on the period of time of subsequently freeze-drying after loading the cells, or after washing loaded cells, but also, in some cases, on whether or not the inert substance and/or protein has been admixed with the solute in the salt solution, and/or, in other cases, the quantity of the inert substance and/or the protein that has been added. Curve 1110 represents hemolysis vs. incubation time (hours) when incubating cells with 300 mOsm PBS. Curve 1114 represents hemolysis vs. incubation time (hours) when incubating cells with 15% HES. Curve 1118 represents hemolysis vs. incubation time (hours) when incubating cells with 30% HES. Curve 1122 represents hemolysis vs. incubation time (hours) when incubating cells with 2.5% HSA. Curve 1126 represents hemolysis vs. incubation time (hours) when incubating cells with 5% HSA. Curve 1130 represents hemolysis vs. incubation time (hours) when incubating cells with 2.5% HAS and 15% HES. Thus, adding the inert substance and/or the protein, particularly in the quantities of 15% (i.e., HES) and 2.5% (i.e., HSA), to the incubation solution assists in further reducing hemolysis. As will be further explained below, the addition of the inert substance and/or the protein, both in the freeze-drying buffer and in the rehydration buffer, has significantly improved cell drying (e.g., freeze-drying) and rehydration such that rehydrated cells have normal discoid morphology. Example 8 below provides the more specific testing conditions and parameters which produced the graphical illustrations of FIG. 11.

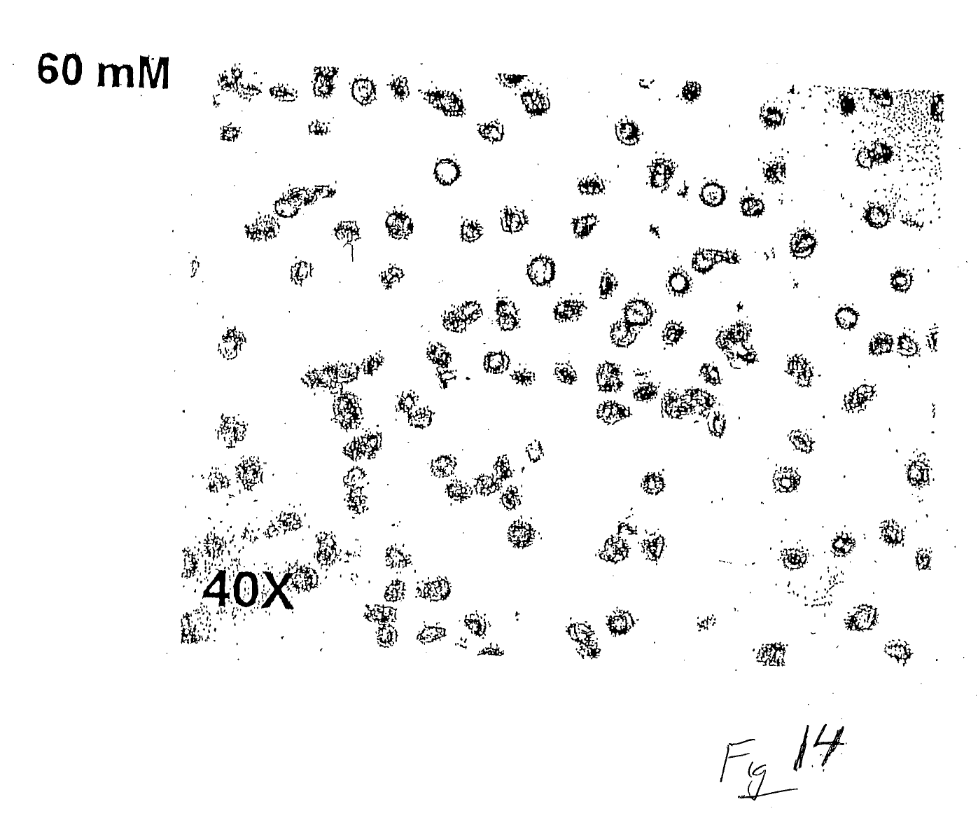

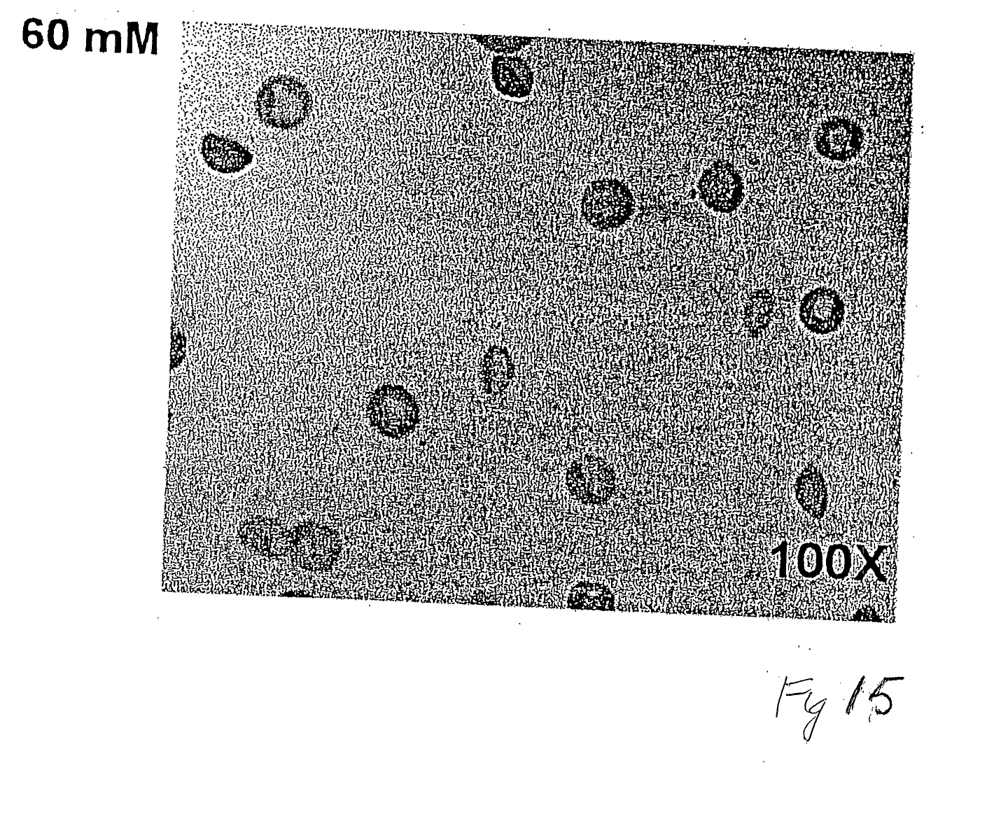

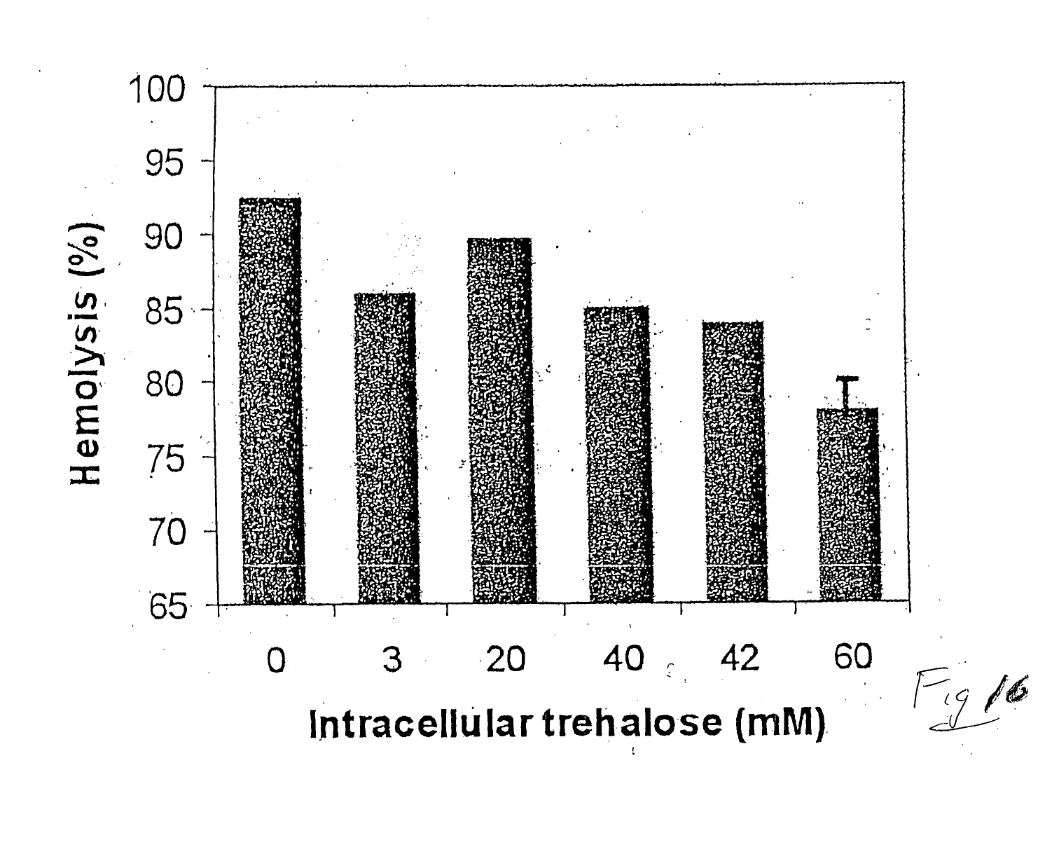

Referring now to FIGS. 12-15, there are seen pictures of cells (e.g., erythrocytic cells) which illustrate that intracellular trehalose, more particularly intracellular trehalose along with intracellular inert substance and protein, improve the survival of rehydrated cells. FIG. 12 is a 40× picture of rehydrated erythrocytic cells having no intracellular trehalose prior to freeze-drying. FIG. 13 is a 40× picture of rehydrated erythrocytic cells having 3 mM intracellular trehalose, after initially trehalose-loading and freeze-drying the cells in 300 mM trehalose/100 mOsm PBS, 15% HES and 2.5% HSA. FIG. 14 is a 40× picture of rehydrated erythrocytic cells having 60 mM intracellular trehalose, after initially trehalose-loading and freeze-drying the cells in 300 mM trehalose/100 mOsm PBS, 15% HES and 2.5% HSA. FIG. 15 is a 100× picture of rehydrated erythrocytic cells having 60 mM intracellular trehalose, after initially trehalose-loading and freeze-drying the cells in 300 mM trehalose/100 mOsm PBS, 15% HES and 2.5% HSA. Example 9 below provides the more specific testing conditions and parameters which produced the graphical illustrations of FIGS. 12-15.

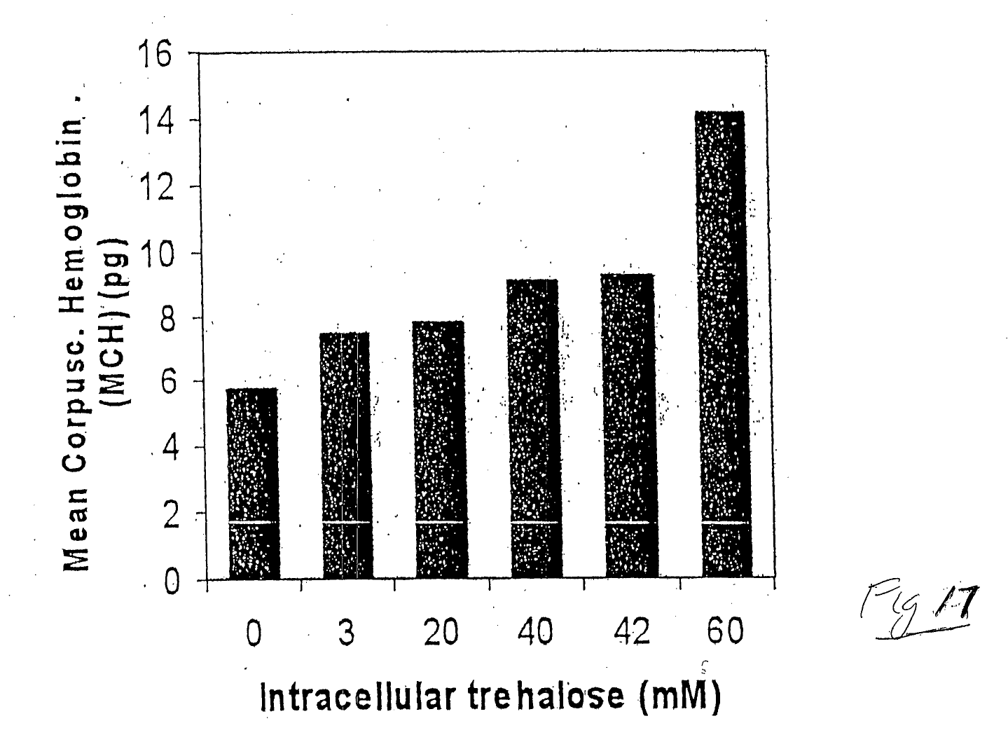

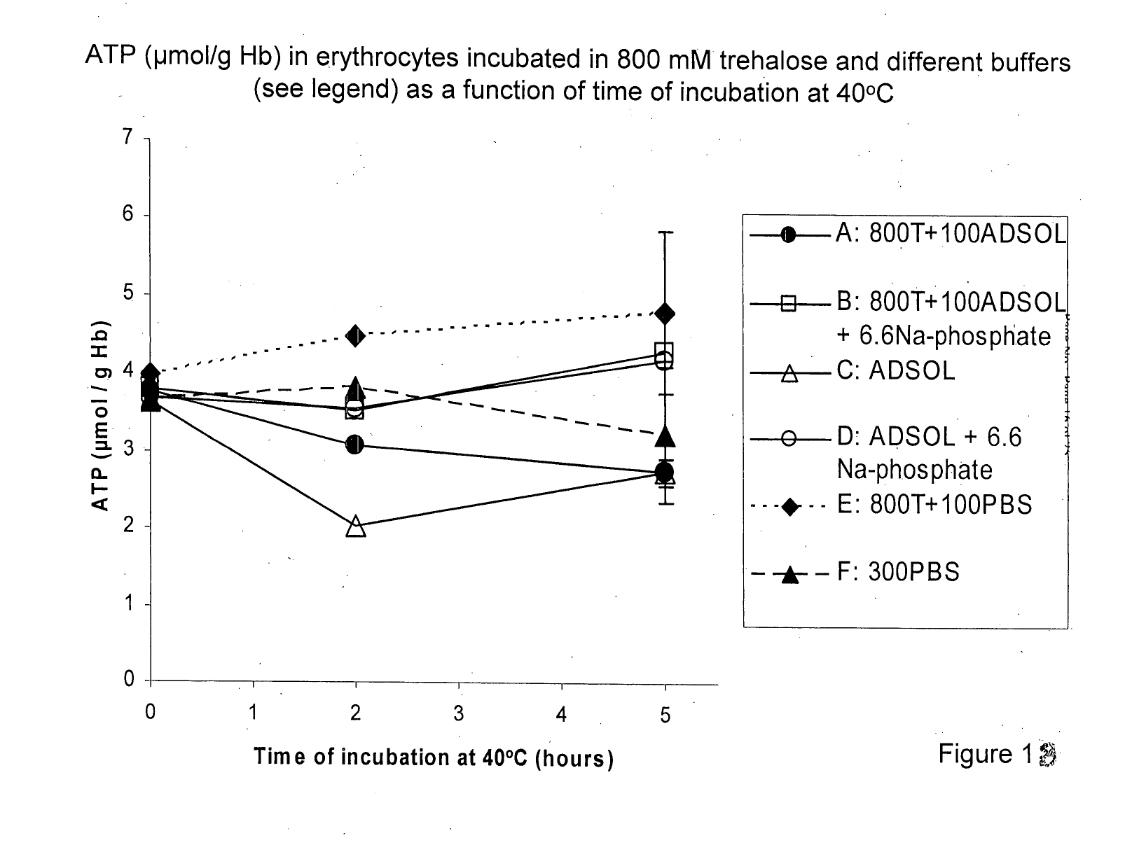

The concentration of intracellular trehalose in rehydrated cells is also important for subsequent stabilization of the rehydrated cells. Referring now to FIG. 17, there is seen a graph of mean corpuscular hemoglobin of trehalose-loaded, freeze-dried and rehydrated erythrocytic cells as a function of intracellular trehalose concentration (mM). FIG. 17 illustrates that as the concentration of intracellular trehalose increases for rehydrated erythrocytic cells, the mean corpuscular hemoglobin (MCH, the amount of hemoglobin found in intact erythrocytic cells) also increases for rehydrated erythrocytic cells. Example 11 below provides the more specific testing conditions and parameters which produced the graphical illustrations of FIG. 17. An intracellular trehalose concentration (mM) of about 40 mM and (or to) about 42 mM produces an MCH of about 9 (pg). When the intracellular trehalose concentration (mM) increases to about 60 mM, the MCH precipitously increases to above about 10 (pg), more specifically above or greater than about 14 (pg). Thus, embodiments of the present invention include loading cells with an effective amount of solute for stabilizing cells. An effective amount of a solute is greater than about 50 mM, such as 60 mM or above. Furthermore, as previously indicated, as intracellular concentration increases, there is a significant decrease in the percent (%) hemolysis, to an extent of less than about 10%, and even less than about 5%, as broadly illustrated in FIG. 16 which is a graph of hemolysis(%) of trehalose loaded erythrocytic cells as a function of intracellular trehalose concentration (mM), graphically illustrating the effect of cytoplasmic trehalose on the survival of rehydrated erythrocytic cells. Example 10 below provides the more specific testing conditions and parameters which produced the graphical illustrations of FIG. 16. As cytoplasmic intracellular trehalose approaches a concentration of about 100 mM, % hemolysis falls to below about 10%.

The following protocol has been discovered as yielding significant survival of freeze-dried cells. The loading buffer comprised about 800 mM trehalose in a salt solution of about 100 mOsm PBS. The incubation time was about 16 hours at a temperature of about 35° C. After the cells were loaded, they were subsequently washed in a washing buffer comprising about 300 mM trehalose in a salt solution of about 100 mOsm PBS. Within about 3 hours after washing the loaded cells, the wash loaded cells were freeze-dried in freeze-drying buffer comprising about 300 mM trehalose, about 100 mOsm PBS, about 2.5% by wt. HSA, and about 15% by wt. HES. After freeze-drying, the cells had about 75 mM trehalose, about 25 mOsm PBS, about 0.6% by wt. HSA and about 4.0% by wt. HES left in the cells. In various embodiments of the invention for producing maximal survival of the cells, the dried cells comprise from about 25 mM to_about 300 mM trehalose, from about 5 mOsm to about 100 mOsm osmolarity for the salt solution, from about 0.1% by weight to about 2.5% by weight of the protein, and from about 1.0% by weight to about 15.0% by weight of the inert substance; and preferably from about 60 mM to about 80 mM trehalose, from about 10 mOsm to about 40 mOsm PBS, from about 0.3% by weight to about 9.0% by weight albumin, and about 1.0% by weight to about 4.0% by weight starch. The freeze-dried cells were then reconstituted at about 37° C. for about 10 minutes in a rehydration buffer comprising about 188 mM trehalose, about 100 mOsm PBS, about 2.5% by wt. HSA and about 15.0% by wt. HES. After rehydration, less than about 5% of the cells were lysed. Example 12 below also provides the conditions and parameters which produced the foregoing protocol yielding less than 5% hemolysis.

In other embodiments of the present invention the solute solution includes one or more of the following: a monosaccharide, a monosaccharide polyol, a cell metabolite-controlling agent (e.g., a heterocyclic compound, such as adenine), and optionally, a salt. The solute solution for these embodiments of the present invention may be used for any suitable purpose, such as a loading or incubating solution, or as a drying solution, or a rehydrating solution. When the solute solution is used for loading a solute, the solute solution may also comprise the solute, and optionally, a buffering-salt chemical or compound. The solute solution for these embodiments of the invention may be used for any biological sample, particularly for erythrocytes (i.e., red blood cells).

For various embodiments of the present invention the solute solution may comprise at least about 0.1 weight % of a monosaccharide (e.g., from about 0.1 weight % to about 20.0 weight % of the monosaccharide), at least about 0.05 weight % of a monosaccharide polyol (e.g., from about 0.05 weight % to about 15.0 weight % of the monosaccharide polyol), at least about 0.02 weight % of a cell metabolite-controlling agent (e.g., from about 0.02 weight % to about 8.0 weight % of the cell metabolite-controlling agent), and, optionally at least about 0.02 weight % of a salt (e.g., from about 0.02 weight % to about 15.0 weight % of the salt).

For further various embodiments of the present invention the solute solution may comprise from about 0.2 weight % to about 7.24 weight % (e.g., from about 10 mM to about 400 mM) of a monosaccharide, from about 0.018 weight % to about 3.64 weight % (e.g., from about 1 mM to about 200 mM) of a monosaccharide polyol, from about 0.00135 weight % to about 0.675 weight % (e.g., from about 0.1 mM to about 50 mM) of a cell metabolite-controlling agent (e.g., adenine), and, optionally from about 0.058 weight % to about 2.92 weight % (e.g., from about 10 mM to about 500 mM) of a salt (e.g., NaCl); more preferably from about 0.905 weight % to about 3.62 weight % (e.g., from about 50 mM to about 200 mM) of a monosaccharide, from about 0.182 weight % to about 1.82 weight % (e.g., from about 10 mM to about 100 mM) of a monosaccharide polyol, from about 0.0068 weight % to about 0.135 weight % (e.g., from about 0.5 mM to about 10 mM) of a cell metabolite-controlling agent (e.g., adenine), and, optionally from about 0.292 weight % to about 1.752 weight % (e.g., from about 50 mM to about 300 mM) of the salt; and most preferably from about 1.45 weight % to about 2.72 weight % (e.g., from about 80 mM to about 150 mM) of a monosaccharide, from about 0.36 weight % to about 1.10 weight % (e.g., from about 20 mM to about 60 mM) of a monosaccharide polyol, from about 0.014 weight % to about 0.068 weight % (e.g., from about 1 mM to about 5 mM) of a cell metabolite-controlling agent (e.g., adenine), and, optionally from about 0.58 weight % to about 1.17 weight % (e.g., from about 100 mM to about 200 mM) of a salt (e.g., NaCl).

For other various embodiments of the present invention where the solute solution is used for loading a solute, the solute solution may comprise at least about 5.0 weight % of a solute (e.g., from about 5.0 weight % to about 70.0 weight % of the solute), at least about 0.1 weight % of a monosaccharide (e.g., from about 0.1 weight % to about 20.0 weight % of the monosaccharide), at least about 0.05 weight % of a monosaccharide polyol (e.g., from about 0.05 weight % to about 15.0 weight % of the monosaccharide polyol), at least about 0.02 weight % of a cell metabolite-controlling agent (e.g., from about 0.02 weight % to about 8.0 weight % of the cell metabolite-controlling agent), and, optionally at least about 0.02 weight % of a salt (e.g., from about 0.02 weight % to about 15.0 weight % of the salt), and, optionally further at least about 0.01 weight % of a buffering-salt chemical or compound (e.g., from about 0.01 weight % to about 5 weight % of the buffering-salt chemical or compound (e.g., KH₂PO₄: K₂HPO₄, 1:2).

For further other embodiments of the invention where the solute solution is used for loading a solute, the solute solution may comprise from about 15.12 weight % to about 49.14 weight % (e.g., from about 400 mM to about 1300 mM) of a solute, from about 0.018 weight % to about 1.81 weight % (e.g., from about 1 mM to about 100 mM) of a monosaccharide, from about 0.018 weight % to about 1.82 weight % (e.g., from about 1 mM to about 100 mM) of a monosaccharide polyol, from about 0.000135 weight % to about 0.135 weight % (e.g., from about 0.001 mM to about 10 mM) of a cell metabolite-controlling agent (e.g., adenine), and, optionally from about 0.0292 weight % to about 0.584 weight % (e.g., from about 5 mM to about 100 mM) of a salt (e.g., NaCl), and, optionally further from about 0.01 weight % to about 1.0 weight % of a buffering-salt chemical or compound (e.g., from about 0.1 mM to about 30 mM, and for a mixture of KH₂PO₄ and K₂HPO₄, from about 0.014 weight % KH₂PO₄ and from about 0.017 weight % K₂HPO₄ to about 0.40 weight % KH₂PO₄ and to about 0.52 weight % K₂HPO₄, respectively); more preferably from about 18.90 weight % to about 41.58 weight % (e.g., from about 500 mM to about 1100 mM) of a solute, from about 0.091 weight % to about 1.27 weight % (e.g., from about 5 mM to about 70 mM) of a monosaccharide, from about 0.055 weight % to about 0.91 weight % (e.g., from about 3 mM to about 50 mM) of a monosaccharide polyol, from about 0.00068 weight % to about 0.068 weight % (e.g., from about 0.05 mM to about 5 mM) of a cell metabolite-controlling agent (e.g., adenine), and, optionally from about 0.058 weight % to about 0.41 weight % (e.g., from about 10 mM to about 70 mM) of a salt (e.g., NaCl), and, optionally further from about 0.015 weight % to about 0.90 weight % of a buffering-salt chemical or compound (e.g., from about 0.5 mM to about 20 mM, and for a mixture of KH₂PO₄ and K₂HPO₄, from about 0.0068 weight % KH₂PO₄ and from about 0.0087 weight % K₂HPO₄ to about 0.27 weight % KH₂PO₄ and to about 0.35 weight % K₂HPO₄, respectively); and most preferably from about 22.68 weight % to about 37.80 weight % (e.g., from about 600 mM to about 1000 mM) of a solute, from about 0.18 weight % to about 0.91 weight % (e.g., from about 10 mM to about 50 mM) of a monosaccharide, from about 0.091 weight % to about 0.73 weight % (e.g., from about 5 mM to about 40 mM) of a monosaccharide polyol, from about 0.0011 weight % to about 0.027 weight % (e.g., from about 0.08 mM to about 2 mM) of a cell metabolite-controlling agent (e.g., adenine), and, optionally from about 0.12 weight % to about 0.35 weight % (e.g., from about 20 mM to about 60 mM) of a salt (e.g., NaCl), and, from about 0.02 weight % to about 0.80 weight % of a buffering-salt chemical or compound (e.g., from about 1.0 mM to about 10 mM, and for a mixture of KH₂PO₄ and K₂HPO₄, from about from about 0.014 weight % KH₂PO₄ and from about 0.017 weight % K₂HPO₄ to about 0.14 weight % KH₂PO₄ and to about 0.17 weight % K₂HPO₄, respectively).

The monosaccharide may be any suitable monosaccharide, preferably glucose, particularly because glucose is presently the most cost effective cellular source of energy. However, the spirit and scope of the present invention would include other suitable monosaccharides.

The monosaccharide polyol may be any suitable monosaccharide polyol (e.g., a sugar alcohol), such as, by way of example only, one selected from the group including mannitol, sorbitol, maltitol, xylitol and lactitol, and mixtures thereof. A preferred monosaccharide polyol comprises mannitol.

The cell metabolite-controlling agent may be any suitable chemical or compound which is capable of controlling (e.g., interacting with and/or maintaining) the level of metabolites in the biological sample(s), particularly the metabolites adenosine-3-phosphate (ATP) and 2,3-diphosphoglycerate (2,3-DPG). ATP level correlates with the efficiency of the glycolic pathway which is the major biochemical pathway in erythrocytes. The polyanion 2,3-DPG binds to the central cavity of the hemoglobin tetramer and modulates the affinity of hemoglobin for oxygen. It is important for the oxygen carrying capacity of hemoglobin. The cell metabolite-controlling agent comprises a heterocycle compound. Preferably, the cell metabolite-controlling agent comprises adenine.

Adenine is a heterocyclic base compound which comprising an integral part of adenosine-3-phosphate (ATP), a high-energy phosphate compound in biological samples (e.g., erythrocytes) that phosphorylates cellular molecules. When an extracellular medium is supplemented with adenine, it crosses the cellular membrane and becomes part of the synthesis of adenosine-3-phosphate (ATP). ATP is important because it has the ability to phosphorylate or to add a phosphate group to other molecules, such as proteins. This transfer of phosphate groups allows energy to be released. It is this energy that is used by the biological samples, including cells and living organisms. This is why ATP and its heterocyclic base adenine are important.

2,3-diphosphoglycerate (i.e., 2,3-DPG) does not comprise adenine. 2,3-DPG is a phosphorylated sugar acid which is present in all cells. However, erythrocytes contain considerably greater amounts of 2,3-DPG, a quantity approximately equimolar with the concentration of hemoglobin itself. It is the interaction with hemoglobin that accounts for its special role in the erythrocytes. 2,3-DPG is important because synthesis of 2,3-DPG is a measure for the activity of the special side pathway that branches from the main glycolytic pathway.

Levels of ATP and DPG may be measured or assayed in biological samples (e.g., in cells) by any suitable means or procedure. By way of example only, levels of ATP and DPG may be measured or assayed in biological samples (e.g., in cells) by using Sigma Diagnostic (Sigma Aldrich Chemicals, St. Louis, Mo.) assay kits (cat. # for ATP assay 366 and 366-A, and for 2,3-DPG assay 35-A) designed for determination of ATP and 2,3-DPG in biological samples (e.g., red blood cells). By further way of example only, biological samples (e.g., cells) may be lysed using 12% and 8% trichloracetic acid for ATP and 2,3-DPG determination, respectively. ATP may be quantified enzymatically by catalyzing ATP to ADP in the presence of 3-phosphoglycerate (3-PGA) using phosphoglycerate phosphokinase (PGK) and the resulting 1,3-diphosphoglycerate induced oxidation of NADH to NAD using glyceraldehyde phosphate dehydrogenase (GAPD) present in the assay media. The level of NADH may be monitored spectrophotometrically at 340 nm which corresponds directly with the ATP level. 2,3-DPG may be hydrolyzed by 2,3-DPG phosphatase to 3-PGA and inorganic phosphorus in the presence of 2-phosphoglycolic acid as a stimulator. 3-PGA reacts with ATP in the presence of PGK and the subsequent steps follow the above protocol used for ATP quantification. In this case the level of NADH is directly proportional to the concentration of 3-PGA, which in turn corresponds to the concentration of 2,3-DPG in the reaction media. ATP and 2,3-DPG concentrations may be expressed in μmol/g hemoglobin.

The salt and the buffering-salt chemical or compound may be, respectively, any suitable salt and any suitable buffering-salt chemical or compound which is capable of controlling or producing a saline or buffering effect. The salt may comprise KCl, NaCl, CaCl₂, etc. The buffering-salt chemical or compound may comprise sodium phosphate, potassium phosphate, sodium citrate, potassium citrate, etc. The salt and the buffering-salt chemical or compound preferably comprises NaCl and potassium phosphate, respectively.

For various embodiments of the invention, the buffering-salt chemical or compound may comprise 6.6 mM K-phosphate pH 7.2 sold under Kit PBS-1, catalog number of the product I-9773, by the manufacturer, Sigma-Aldrich of St. Louis, Mo. This K-phosphate product comprises a mixture of KH₂PO₄ (monobasic) and K₂HPO₄ (dibasic) in a volume ratio of about 1:2. This ratio provides a desired pH of about 7.2. A recipe for preparing 6.6 mM K-phosphate comprises mixing stocks of 1 M KH₂PO₄ and 1 M K₂HPO₄ to preferably produce 100 mM KH₂PO₄/K₂HPO₄. For this purpose, 33.6 ml of 1 M KH₂PO₄ is mixed with 68.4 ml of 1 M K₂HPO₄. The resulting mixture comprises 100 mM K-phosphate with a pH of about 7.2.

When the monosaccharide comprises glucose and the monosaccharide polyol comprises mannitol and the cell metabolite-controlling agent comprises adenine, such constituents for the solute solution may be provided under the commercial available product sold under the trademark ADSOL by the Baxter Healthcare Corporation of Deerfield, Ill. ADSOL comprises 111 mM glucose, 2 mM adenine, 154 mM NaCl and 41 mM mannitol, and has used commercially by blood banks as an erythrocyte storing media, but not as a biological sample (e.g., erythrocytes) loading, drying, or rehydrating media. A solute solution comprising one or more of glucose, adenine, NaCl and mannitol (e.g., 111 mM glucose, 2 mM adenine, 154 mM NaCl and 41 mM mannitol) may be used for any purpose within any framework for various embodiments of the present invention, including as a loading buffer, as a drying buffer, or as a rehydrating buffer.

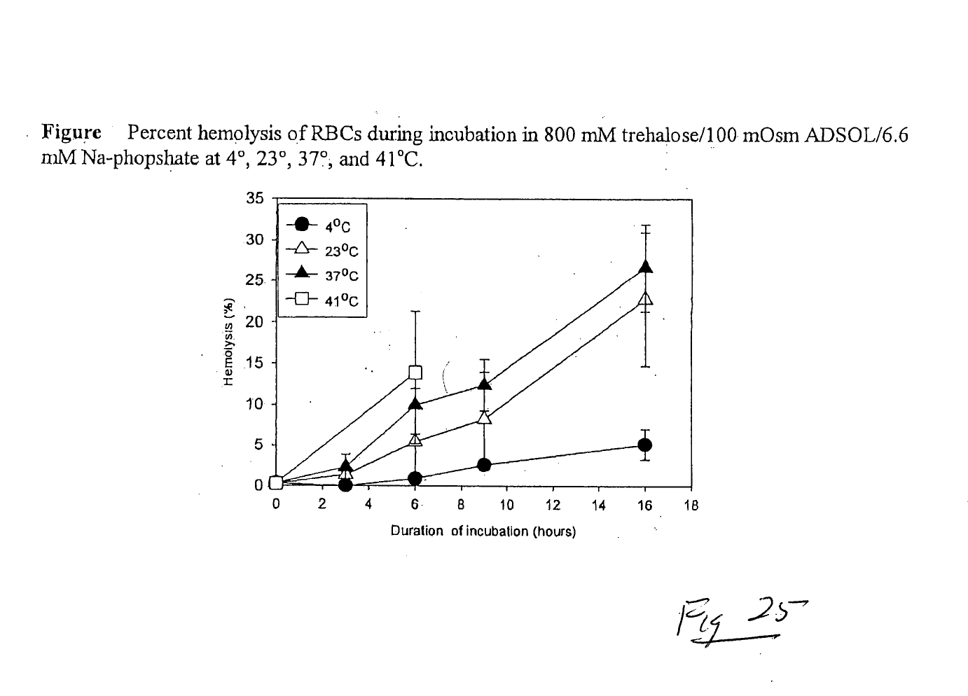

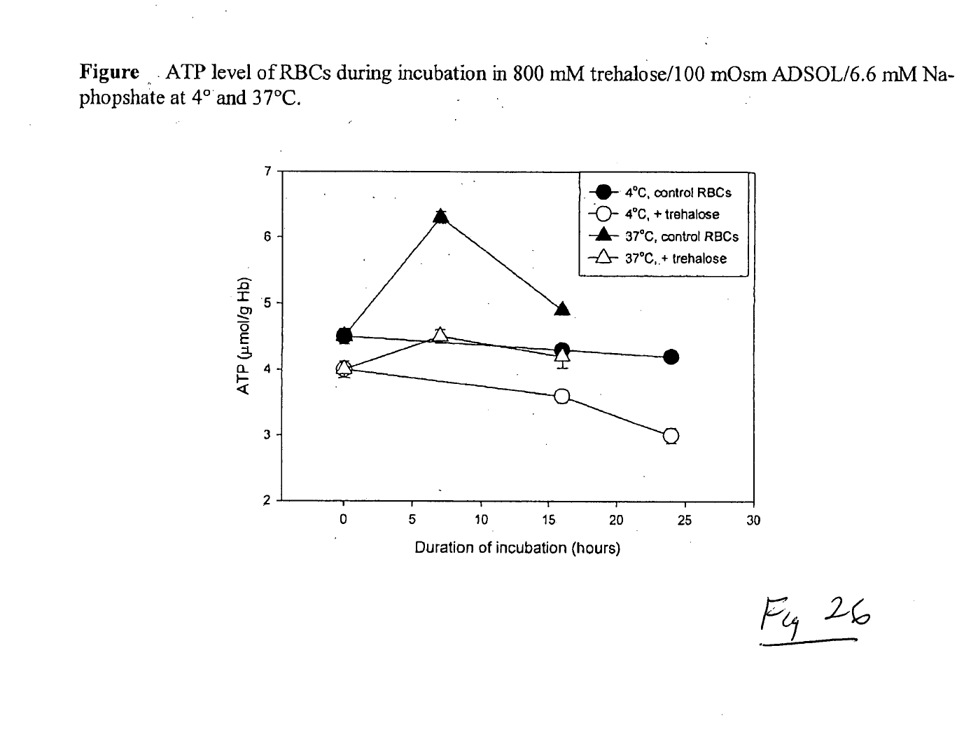

Referring now to FIG. 24 there is seen a graph of trehalose uptake (e.g., cytoplasmic trehalose concentration mM) as a function of time of incubation in a solute solution comprising 800 mM trehalose, 100 mOsm ADSOL, and 6.6 mM K-phosphate (pH 7.2). 100 mOsm ADSOL comprises 24 mM glucose, 0.43 mM adenine, 33.3 mM NaCl, and 8.9 mM mannitol. FIG. 25 is a graph of percent hemolysis of erythrocytes (RBCs) at 4° C., 23° C., 37° C. and 41° C. as a function of time of incubation in a solute solution comprising 800 mM trehalose, 100 mOsm ADSOL, and 6.6 mM K-phosphate (pH 7.2). Example 13 below provides the more specific testing conditions and parameters which produced the graphical illustrations of FIGS. 24 and 25.

FIG. 24 illustrates that the efficiency of loading increases with time and temperature of incubation reaching an optimal level at around about 37° C. (e.g., from about 35° C. to about 39° C.), above which there is no further essential or significant increase in trehalose uptake while hemolysis increases as illustrated in FIG. 25 which reflects that hemolysis precipitously increases after about nine(9) hours of incubation in the loading solute solution. As shown in FIG. 24 nine(9) hours of incubation at 4° C., 23° C., and 37° C. results in about 10 mM, about 22 mM and about 60 mM cytoplasmic trehalose, respectively, while the percent hemolysis during this nine(9) hour loading time was about 2%, about 7%, and about 12% for the erythrocytes, respectively. Based on the data reflected in FIGS. 24 and 25 one of the favorable loading conditions versus the amount of hemolysis would be loading at an incubation temperature at about 37° C. (i.e., a temperature from between about 35° C. to about 39° C.) for about nine(9) hours (e.g., from about 5 hours to about 10 hours, such as from about 6 hours to about 9 hours or from about 7 hours to about 9 hours). Under these loading conditions the trehalose (mM) to incubation (hours) loading gradient is about 1.25 (e.g., from about 1.0 to about 1.5, or from about 1.10 to about 1.35). Under these loading conditions the biological-sample hemolysis (%) to incubation (hours) hemolysis gradient is about 0.25 (e.g., from about 0.1 to about 0.5, or from about 0.15 to about 0.35, or from about 0.20 to about 0.30). At a loading incubation temperature of about 23° C., the trehalose (mM) to incubation (hours) loading gradient is about 0.75. Also at a loading incubation temperature of about 23° C., the biological-sample hemolysis (%) to incubation (hours) hemolysis gradient is about 0.38. Thus, the trehalose (mM) to incubation (hours) loading gradient at about 37° C. increases from the trehalose (mM) to incubation (hours) loading gradient at about 23° C. (i.e., increases from about 0.38 to about 0.75), while the biological-sample hemolysis (%) to incubation (hours) hemolysis gradient at about 37° C. decreases from the hemolysis (%) to incubation (hours) hemolysis gradient at about 23° C. (i.e., decreases from 0.38 to about 0.25).

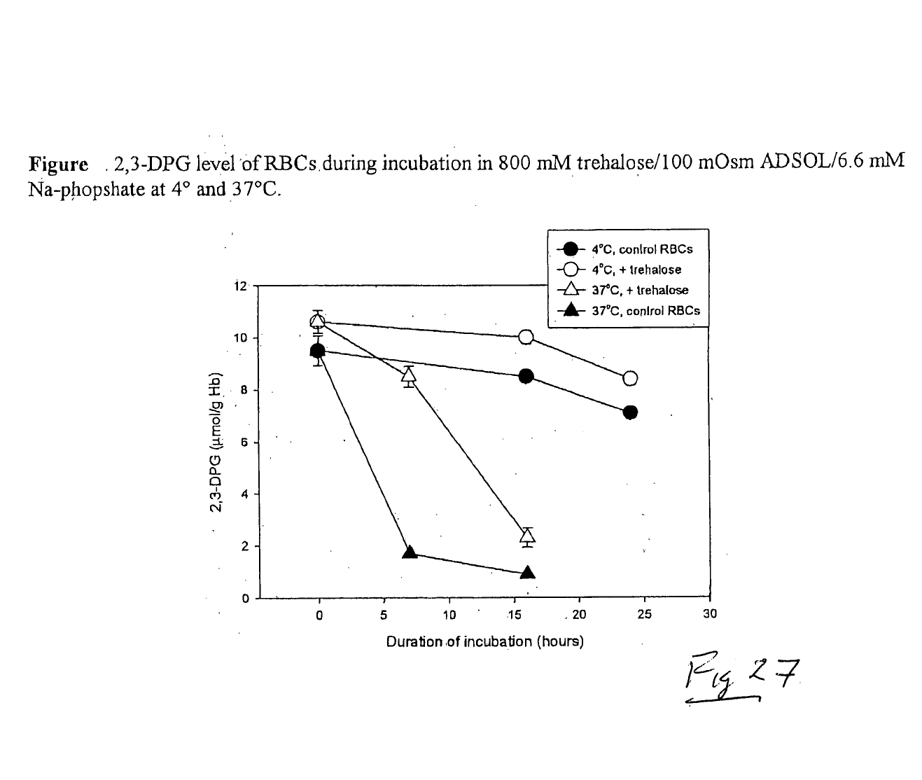

For various embodiments of the present invention, in parallel with and/or simultaneously with following and/or monitoring the quantity (mM) of trehalose loading and biological sample (i.e., cell) hemolysis, the levels of ATP and 2,3-DPG are preferably followed or monitored. Referring now to FIG. 26 there is seen a graph of ATP (μmol/g Hb) levels (or quantities) for control RBCs and for trehalose-loaded RBCs during incubation in 800 mM trehalose/100 mOsm ADSOL/6.6 mM K-phosphate at 4° and 37° C. In FIG. 27 there is seen a graph of 2,3-DPG (μmol/g Hb) levels (or quantities) for control RBCs and for trehalose-loaded RBCs during incubation in 800 mM trehalose/100 mOsm ADSOL/6.6 mM K-phosphate at 4° and 37° C. Example 14 below provides the more specific testing conditions and parameters which produced the graphical illustrations of FIGS. 26 and 27.

Both ATP and 2,3-DPG metabolites are essential for a biological sample (e.g., a cell) viability. The normal level of ATP in vivo is between about 3.65 and about 4.45 μmol/gHb, and that of 2,3-DPG is about 13±3 μmol/gHb. In FIG. 26 there is seen the level or quantity of ATP remaining between about 3.65 and about 4.45 μmol/gHb, the level for in vivo, both for the control RBCs and for the RBCs (trehalose-loaded) being incubated/loaded in a solute solution having trehalose. The level or quantity of ATP μmol/gHb for RBCs (trehalose-loaded) being incubated/loaded in a solute solution having trehalose increases (e.g., increases from about 4.0 μmol/gHb to about 4.5 μmol/gHb) when incubating at about 37° C. (i.e., at a temperature ranging from between about 35° C. to about 39° C.) from about 0 hours to about 10 hours (i.e., from about 0 hours to about 7 hours). Thus, for various embodiments of the invention, a positive ATP μmol/gHb to incubating duration (hours) gradient is maintained during the loading of a biological sample (e.g., erythrocytes) with a solute, such as trehalose.

In FIG. 27 it is seen that the μmol/gHb level of 2,3-DPG precipitously declines (i.e., declines from about 9.5 μmol/gHb to about 1.2 μmol/gHb) when control RBCs are incubated at about 37° C. for about 7 hours. However, during 7 hours incubation at about 37° C. in a trehalose containing solution, the μmol/gHb level of 2,3-DPG remains high (about 8.5 μmol/gHb). These results show that loading at about 37° C. (i.e., at a temperature ranging from between about 35° C. to about 39° C.) for about 7 hours (i.e., from about 0 hours to about 10 hours, such as from about 4 hours to about 9 hours, or from about 4 hours to about 8 hours, or from about 6 hours to about 8 hours) provides high μmol/gHb level of 2,3-DPG (while maintaining acceptable μmol/gHb levels of ATP), and that these loading conditions may be used for loading biological samples (e.g., RBCs) with a solute (e.g., trehalose).

The following loading protocol has also been discovered as yielding significant survival of freeze-dried cells. The loading protocol includes incubating the erythrocytic cells in 800 mM trehalose, 100 mOsm ADSOL and 6.6 mM Na-phosphate. ADSOL comprises 111 mM glucose, 2 mM adenine, 154 mM NaCl and 41 mM mannitol. The incubation temperature for loading was between 38 and 41° C., and the time of incubation was 6 hours. This loading procedure yielded lower extent of hemolysis (about 17%), as compared to the hemolysis measured during loading in 800 mM trehalose and 100 mOsm PBS for 16 hours at 37° C. Furthermore, this loading procedure was not accompanied by significant changes in cell morphology. At the same time, the amount of intracellular trehalose was the same as during loading erythrocytes in 800 mM trehalose and 100 mOsm PBS at 37° C. for 16 hours. No washing was applied after termination of the loading step and prior to freeze-drying. Immediately after completing the loading, the cells were mixed gently with the freeze-drying buffer. The final concentration of the freeze-drying buffer was 250 mM trehalose, 20 mOsm ADSOL, 15% HES and 2.5% human serum albumin (HSA). The freeze-dried cells were rehydrated at 37° C. for about 10 min in a rehydration buffer containing 141 mM trehalose, 75 mOsm PBS 11.25% HES and 1.875% HSA.

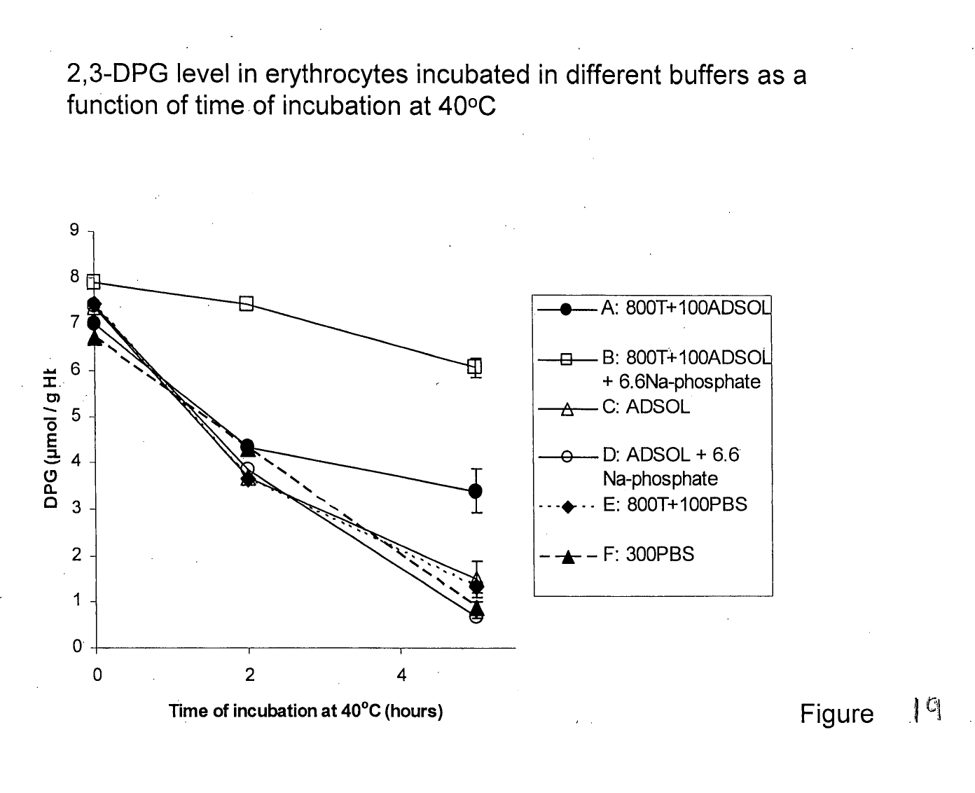

During the loading step, the levels of the following two important metabolites were followed as being essential for cell viability: adenosine-3-phosphate (ATP) and 2,3-diphosphoglycerate (2,3-DPG). ATP level correlates with the efficiency of the glycolic pathway which is the major biochemical pathway in erythrocytes. The polyanion 2,3-DPG binds to the central cavity of the hemoglobin tetramer and modulates the affinity of hemoglobin for oxygen. It is important for the oxygen carrying capacity of hemoglobin. The normal level of ATP in freshly isolated erythrocytes was between 3.65 and 4.45 μmole/g Hb. FIG. 18 shows the ATP level of erythrocytes in buffers with different compositions during 5 hours incubation at 38-41° C. During incubation in 100 mOsm ADSOL and 6.6 mM Na-phosphate (curve D) or in 800 mM trehalose, 100 mOsm ADSOL and 6.6 mM Na-phosphate (curve B), the measured ATP level is very similar to that of freshly isolated erythrocytes. When erythrocytes were incubated in 800 mM trehalose and 100 mOsm PBS, the level of ATP was also as high as in fresh cells (curve E). It was slightly reduced when cells were incubated in 800 mM trehalose and 100 mOsm ADSOL (without Na-phosphate) (curve A), and when the cells were incubated in ADSOL only (462 mOsm) (curve C).

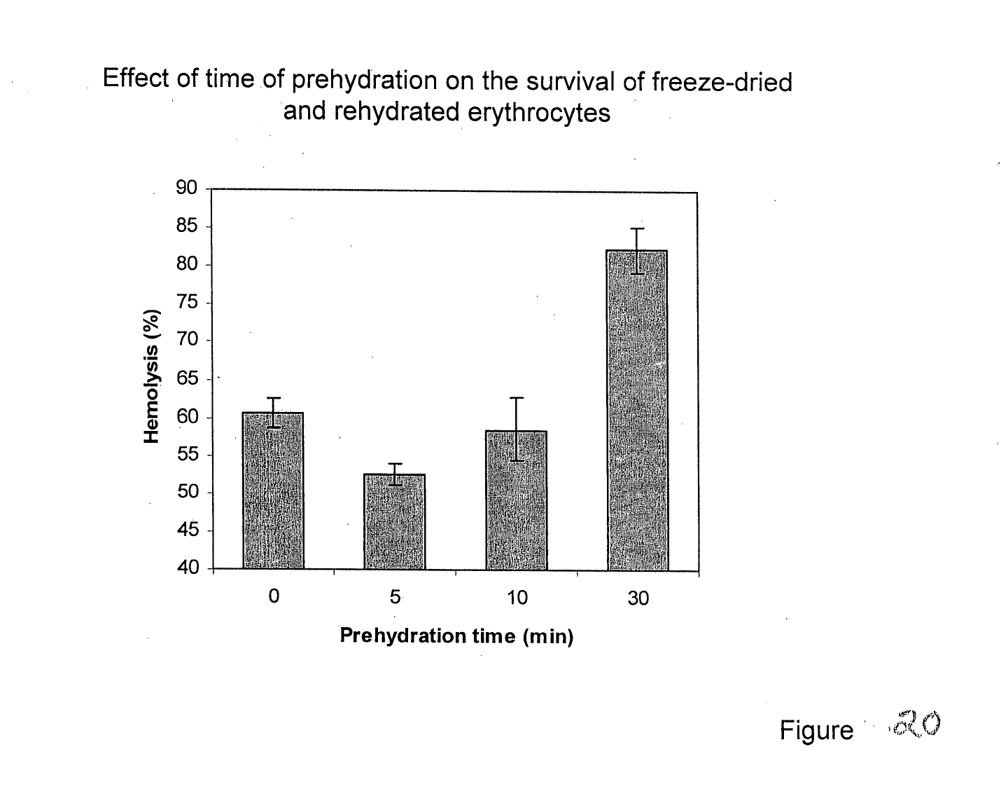

FIG. 19 presents the level of 2,3-DPG during 5 hours incubation at 38-41° C. in buffers with different composition. The normal level of 2,3-DPG in freshly isolated erythrocytes is around 12.8 μmole/g Hb. The highest 2,3-DPG level was observed in cells incubated in 800 mM trehalose, 100 mOsm ADSOL and 6.6 mM Na-phosphate (curve B) and in 800 mM trehalose and 100 mOsm ADSOL (curve A). It was decreased for cells incubated in ADSOL (462 mOsm) (curve C), in ADSOL and 6.6 mM Na-phosphate (curve D), in 800 mM trehalose and 100 mOsm PBS (curve E) and in 300 mOsm PBS (curve F).

On the basis of the data in FIGS. 18 and 19, the incubation medium comprising 800 mM trehalose, 100 mOsm ADSOL and 6.6 mM Na-phosphate provides high levels of ATP and 2,3-DPG.

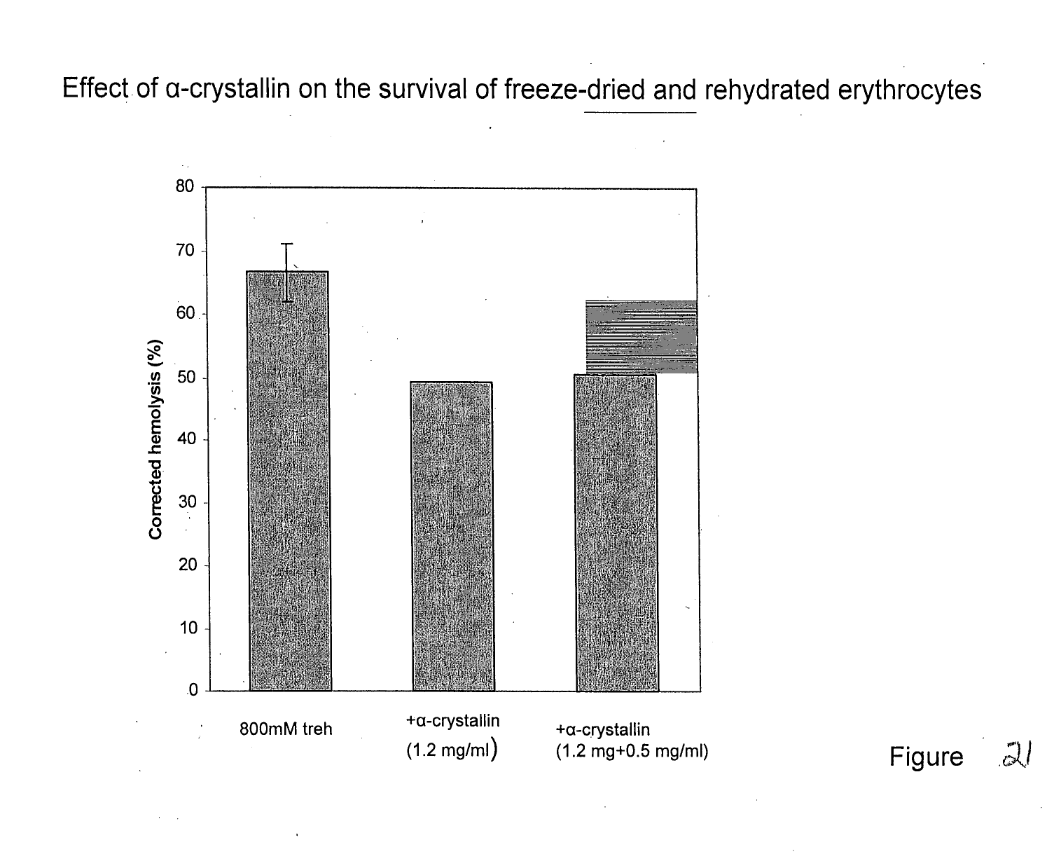

Pre-hydration via exposure to water vapor produces a gradual and more homogenous rehydration of dried biomaterials than direct rehydration. FIG. 20 presents the effect of pre-hydration time on the survival of freeze-dried and rehydrated erythrocytes. Erythrocytic cells were loaded in 800 mM trehalose, 100 mOsm ADSOL and 6.6 mM Na-phosphate at 38-41° C. for 6 hours and were freeze-dried in a buffer with a final concentration of 250 mM trehalose, 20 mOsm ADSOL, 15% HES and 2.5% HSA. Freeze-dried cells were pre-hydrated for various times (between 5 and 30 mins.) and then they were rehydrated at 37° C. for 10 min in a buffer containing 141 mM trehalose, 75 mOsm PBS, 11.25% HES and 1.875% HSA. The data in FIG. 20 show that pre-hydration for 5 min at 37° C. provided the lowest percent of hemolysis.

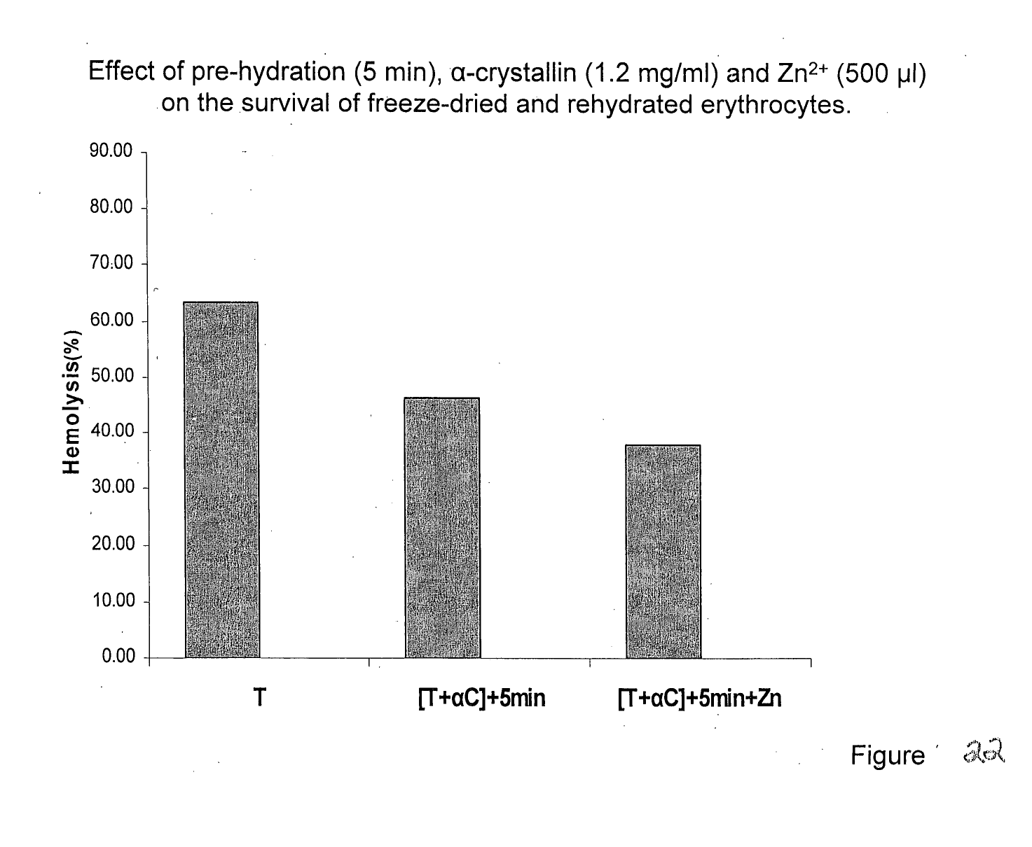

α-crystallin is a member of the small heat shock protein family and is highly abundant in a number of mammalian cell types and tissues. It has been discovered that α-crystallin associates with lipid membranes in vitro and preserves their integrity at high non-lethal temperatures. The results of having studied the effects of α-crystallin on the percent hemolysis are shown in FIG. 21. Cells were loaded in either 800 mM trehalose, 100 mOsm ADSOL, 6.6 mM Na-phosphate (FIG. 21, data set labeled as 800 mM treh) or in 800 mM trehalose, 100 mOsm ADSOL, 6.6 mM Na-phosphate and 1.2 mg/ml α-crystallin (FIG. 21, data set labeled as +α-crystallin 1.2 mg/ml). Cells were subsequently mixed with freeze-drying buffer with final concentration of 250 mM trehalose, 20 mOsm ADSOL, 15% HES and 2.5% HSA and were freeze-dried. After freeze-drying they were directly rehydrated (no pre-hydration) in 141 mM trehalose, 75 mOsm PBS, 11.25% HES and 1.875% HSA. Cells loaded in the presence of 1.2 mg/ml α-crystallin show lower percent hemolysis (49%) in comparison to those loaded without α-crystallin (68%). In the third data set of FIG. 21, along with 1.2 mg/ml α-crystallin, there was 0.5 mg/ml α-crystallin added to the rehydration buffer. The data show that such an increase in the amount of α-crystallin does not result in higher cell survival after rehydration. The conclusion from these data is that α-crystallin improves the survival of freeze-dried and rehydrated erythrocytic cells, as assessed by the decrease in hemolysis from 68% (in cells that have not been loaded in the presence of α-crystallin) to 49% (in cells loaded in the presence of α-crystallin).

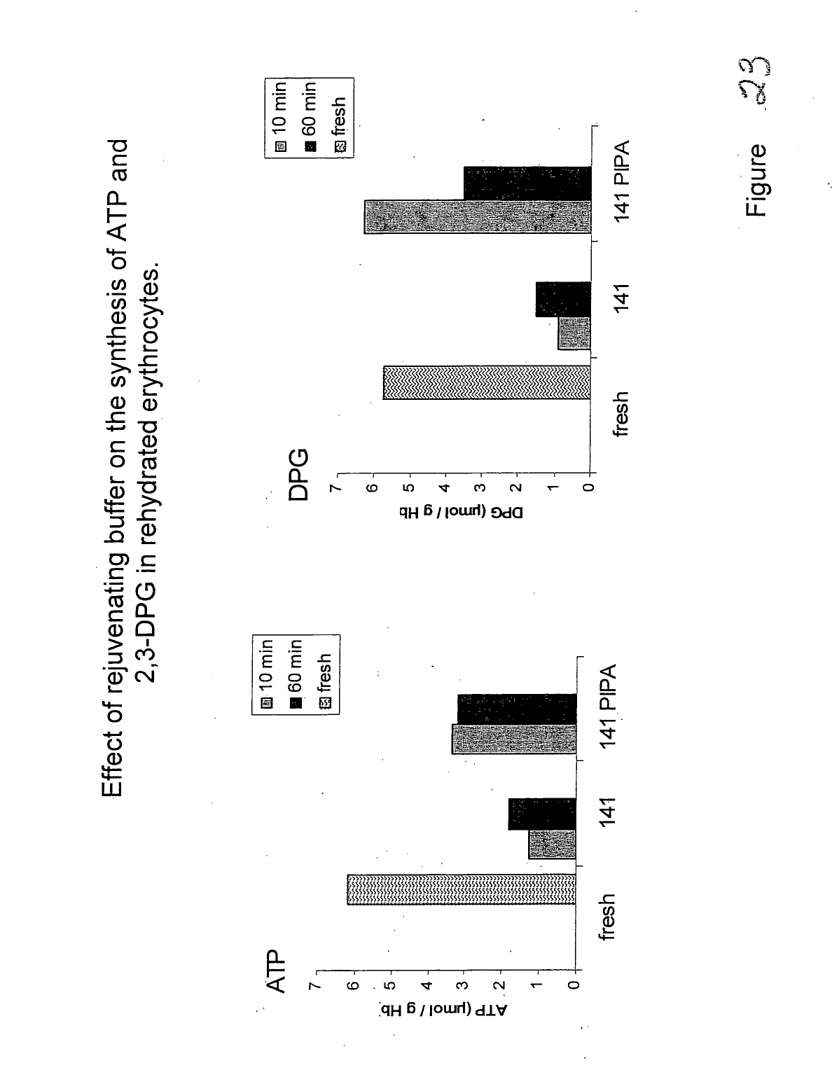

It has been further discovered that when Zn²⁺ ions are added to the rehydration buffer, there is a decrease in the percent hemolysis of rehydrated erythrocytic cells, suggesting that these ions have beneficial effect on cell survival after freeze-drying. Zn²⁺ ions stabilize thermally labile enzymes during drying. Rehydration experiments were performed combining α-crystallin and Zn²⁺ ions, and applying 5 min pre-hydration. Under these conditions, 62% of the cells survived the rehydration step, indicating that the beneficial effect of these treatments is additive. FIG. 22 shows the combined effect of α-crystallin, Zn²⁺ and pre-hydration on the survival of erythrocytic cells. When the cells were loaded in 800 mM trehalose, 100 mOsm ADSOL and 6.6 mM Na-phosphate (FIG. 22, data set labeled as T), freeze-dried in 250 mM trehalose, 20 mOsm ADSOL, 15% HES and 2.5% HSA and directly rehydrated at 37° C. for 10 min in 141 mM trehalose, 75 mOsm PBS, 11.25% HES and 1.875% HSA, the hemolysis was 63%. When the cells were loaded in a buffer containing 800 mM trehalose, 100 mOsm ADSOL, 6.6 mM Na-phosphate and α-crystallin (1.2 mg/ml), freeze-dried in 250 mM trehalose, 20 mOsm ADSOL, 15% HES and 2.5% HAS, pre-hydrated at 37° C. for 5 min, and then fully rehydrated at 37° C. for 10 min in 141 mM trehalose, 75 mOsm PBS, 11.25% HES and 1.875% HSA, the hemolysis was 47% (FIG. 22, data set labeled as [T+αC]+5 min) When cells were loaded in a buffer containing 1.2 mg/ml α-crystallin, 800 mM trehalose, 100 mOsm ADSOL, 6.6 mM Na-phosphate, freeze-dried in 250 mM trehalose, 20 mOsm ADSOL, 15% HES and 2.5% HSA, pre-hydrated for 5 min at 37° C., and rehydrated at 37° C. for 10 min in a buffer containing 500 μM ZnSO₄, 141 mM trehalose, 75 mOsm PBS, 11.25% HES and 1.875% HSA, the hemolysis was only 38% (FIG. 22, data set labeled as [T+αC]+5 min+Zn), giving rise to 62% cell survival.

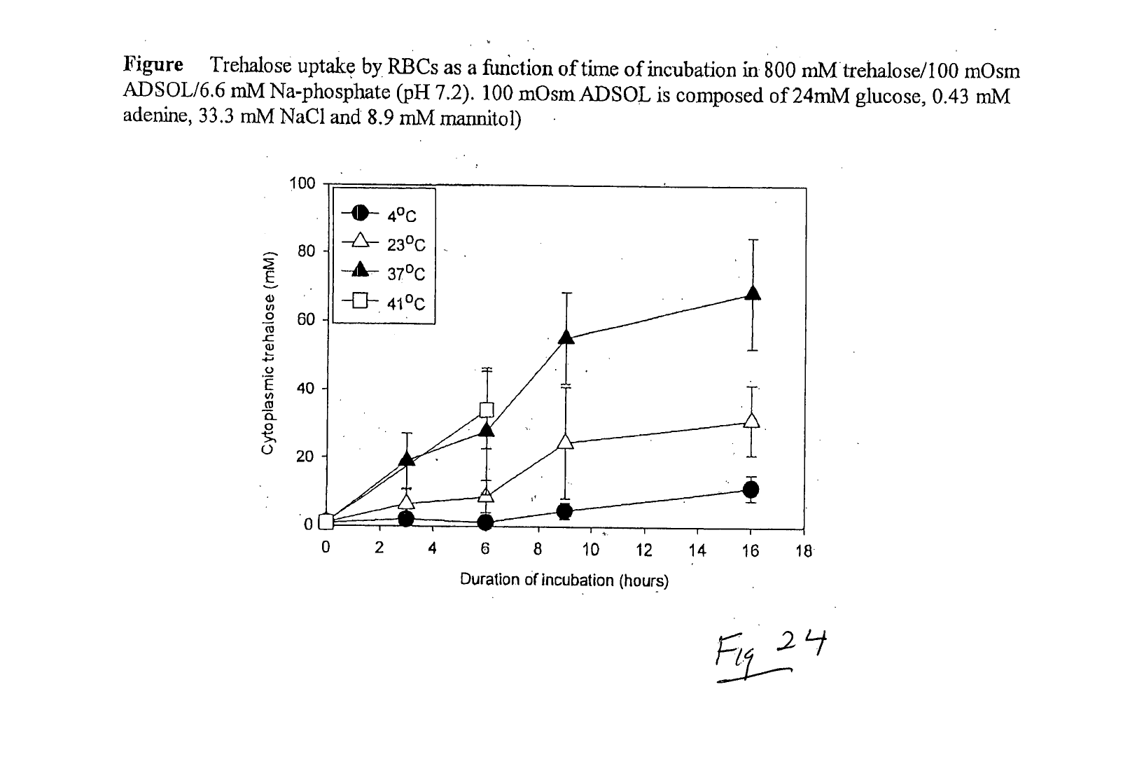

The levels of ATP and 2,3-DPG were followed during rehydration of freeze-dried erythrocytic cells. Incubation of the rehydrated cells in a buffer supplemented with rejuvenation solution led to considerable increase in the ATP and 2,3-DPG synthesis. FIG. 23 shows the levels of the two metabolites during 10 min and 60 min incubation at 37° C. in a rehydration buffer containing 141 mM trehalose, 15% HES, 2.5% HSA and the following rejuvenation supplements: 100 mM pyruvate, 100 mM inosine, 100 mM Na-phosphate and 5 mM adenine. The rejuvenation solution is referred as PIPA. Cells were loaded in 800 mM trehalose, 100 mOsm ADSOL and 6.6 mM Na-phosphate at 38-41° C. for 6 hours. They were freeze-dried in 250 mM trehalose, 20 mOsm ADSOL, 15% HES and 2.5% HSA, and rehydrated at 37° C. for 10 min in 141 mM trehalose, 75 mOsm PBS, 11.25% HES and 1.875% HSA (FIG. 23, data set labeled as 141). Without applying rejuvenation solution, the levels of both ATP and 2,3-DPG are low. However, when such cells were rehydrated in buffer containing 141 mM trehalose, 75 mOsm PBS, 11.25% HES and 1.875% HSA, supplemented with PIPA, the levels of ATP and 2,3-DPG were increased (FIG. 23, data set labeled as 141PIPA). These results show that supplementation of the rehydration medium with 100 mM pyruvate, 100 mM inosine, 100 mM Na-phosphate and 5 mM adenine increases the synthesis of these two vital metabolites and can be applied during reconstitution of freeze-dried erythrocytic cells.

Embodiments of the present invention will be illustrated by the following set forth examples which are being given to set forth the presently known best mode and by way of illustration only and not by way of any limitation. It is to be understood that all materials, chemical compositions and procedures referred to below, but not explained, are well documented in published literature and known to those artisans possessing skill in the art. All materials and chemical compositions whose source(s) are not stated below are readily available from commercial suppliers, who are also known to those artisans possessing skill in the art. All parameters such as concentrations, mixing proportions, temperatures, rates, compounds, etc., submitted in these examples are not to be construed to unduly limit the scope of the invention. Abbreviations used in the examples, and elsewhere, are as follows:
DMSO=dimethylsulfoxide
ADP=adenosine diphosphate
PGE1=prostaglandin El
HES=hydroxy ethyl starch
FTIR=Fourier transform infrared spectroscopy
EGTA=ethylene glycol-bis(2-aminoethyl ether) N,N,N′,N′, tetra-acetic acid
TES=N-tris (hydroxymethyl) methyl-2-aminoethane-sulfonic acid
HEPES=N-(2-hydroxylethyl) piperarine-N′-(2-ethanesulfonic acid)
PBS=phosphate buffered saline
HSA=human serum albumin
BSA=bovine serum albumin
ACD=citric acid, citrate, and dextrose
MβCD=methyl-β-cyclodextrin

### EXAMPLE 1

Washing of Platelets. Platelet concentrations were obtained from the Sacramento blood center or from volunteers in our laboratory. Platelet rich plasma was centrifuged for 8 minutes at 320×g to remove erythrocytes and leukocytes. The supernatant was pelleted and washed two times (480×g for 22 minutes, 480×g for 15 minutes) in buffer A (100 MM NaCl, 10 MM KCl, 10 mM EGTA, 10 mM imidazole, pH 6.8). Platelet counts were obtained on a Coulter counter T890 (Coulter, Inc., Miami, Fla.).

Loading of Lucifer Yellow CH into Platelets. A fluorescent dye, lucifer yellow CH (LYCH), was used as a marker for penetration of the membrane by a solute. Washed platelets in a concentration of 1-2×10⁹ platelets/ml were incubated at various temperatures in the presence of 1-20 mg/ml LYCH. Incubation temperatures and incubation times were chosen as indicated. After incubation the platelets suspensions were spun down for 20× at 14,000 RPM (table centrifuge), resuspended in buffer A, spun down for 20 s in buffer A and resuspended. Platelet counts were obtained on a Coulter counter and the samples were pelleted (centrifugation for 45 s 25 at 14,000 RPM, table centrifuge). The pellet was lysed in 0.1% Triton buffer (10 mM TES, 50 mM KCl, pH 6.8). The fluorescence of the lysate was measured on a Perkin-Elmer LSS spectrofluorimeter with excitation at 428 nm (SW 10 nm) and emission at 530 run (SW 10 nm). Uptake was calculated for each sample as nanograms of LYCH per cell using a standard curve of LYCH in lysate buffer. Standard curves of LYCH, were found to be linear up to 2000 run ml⁻¹.

Visualization of cell-associated Lucifer Yellow. LYCH loaded platelets were viewed on a fluorescence microscope (Zeiss) employing a fluorescein filter set for fluorescence microscopy. Platelets were studied either directly after incubation or after fixation with 1% paraformaldehyde in buffer. Fixed cells were settled on poly-L-lysine coated cover slides and mounted in glycerol.

Loading of Platelets with Trehalose. Washed platelets in a concentration of 1-2 10⁹ platelets/ml were incubated at various temperatures in the presence of 1-20 mg/ml trehalose. Incubation temperatures were chosen from 4° C. to 37° C. Incubation times were varied from 0.5 to 4 hours. After incubation the platelet solutions were washed in buffer A two times (by centrifugation at 14,000 RPM for 20 s in a table centrifuge). Platelet counts were obtained on a coulter counter. Platelets were pelleted (45 S at 14,000 RPM) and sugars were extracted from the pellet using 80% methanol. The samples were heated for 30 minutes at 80° C. The methanol was 10 evaporated with nitrogen, and the samples were kept dry and redissolved in H₂O prior to analysis. The amount of trehalose in the platelets was quantified using the anthrone reaction (Umbreit et al., Mamometric and Biochemical Techniques, 5th Edition, 1972). Samples were redissolved in 3 ml H₂O and 6 ml anthrone reagents (2 g anthrone dissolved in 10M sulfuric acid). After vortex mixing, the samples were placed in a boiling water bath for 3 minutes. Then the samples were cooled on ice and the absorbance was measured at 620 nm on a Perkin Elmer spectrophotometer. The amount of platelet associated trehalose was determined using a standard curve of trehalose. Standard curves of trehalose were found to be linear from 6 to 300 μg trehalose per test tube.

Quantification of Trehalose and LYCH Concentration. Uptake was calculated for each sample as micrograms of trehalose or LYCH per platelet. The internal trehalose concentration was calculated assuming a platelet radius of 1.2 μm and by assuming that 50% of the platelet volume is taken up by the cytosol (rest is membranes). The loading efficiency was determined from the cytosolic trehalose or LYCH concentration and the concentration in the loading buffer.

FIG. 1 shows the effect of temperature on the loading efficiency of trehalose into human platelets after a 4 hour incubation period with 50 mM external trehalose. The effect of the temperature on the trehalose uptake showed a similar trend as the LYCH uptake. The trehalose uptake is relatively low at temperatures of 22° C. and below (below 5%), but at 37° C. the loading efficiency of trehalose is 35% after 4 hours.

When the time course of trehalose uptake is studied at 37° C., a biphasic curve can be seen (FIG. 2). The trehalose uptake is initially slow (2.8×10⁻¹¹ mol/m²s from 0 to 2 hours), but after 2 hours a rapid linear uptake of 3.3×10⁻¹⁰ mol/m²s can be observed. The loading efficiency increases up to 61% after an incubation period of 4 hours. This high loading efficiency is a strong indication that the trehalose is homogeneously distributed in the platelets rather than located in pinocytosed vesicles.

The uptake of trehalose as a function of the external trehalose concentration is shown in FIG. 3, which graphically illustrates the internal trehalose concentration of human platelets versus external trehalose concentration as a function of temperature at a constant incubation or loading time. The uptake of trehalose is linear in the range from 0 to 30 mM external trehalose. The highest internal trehalose concentration is obtained with 50 mM external trehalose. At higher concentrations than 50 mM the internal trehalose concentration decreases again. Even when the loading buffer at these high trehalose concentrations is corrected for isotonicity by adjusting the salt concentration, the loading efficiency remains low. Platelets become swollen after 4 hours incubation in 75 mM trehalose. FIG. 4 graphically illustrates the loading efficiency of trehalose into human platelets as a function of external trehalose concentration.

The stability of the platelets during a 4 hours incubation period was studied using microscopy and flow cytometric analysis. No morphological changes were observed after 4 hours incubation of platelets at 37° C. in the presence of 25 mM external trehalose. Flow cytometric analysis of the platelets showed that the platelet population is very stable during 4 hours incubation. No signs of microvesicle formation could be observed after 4 hours incubation, as can be judged by the stable relative proportion of microvesicle gated cells (less than 3%). The formation of microvesicles is usually considered as the first sign of platelet activation (Owners et al., Trans. Med. Rev., 8, 27-44, 1994). Characteristic antigens of platelet activation include: glycoprotein 53 (gp53, a lysosomal membrane marker), PECAM-1 (platelet endothelial cell adhesion molecule-1, an alpha granule constituent), and P-selection (an alpha granule membrane protein).

### EXAMPLE 2

FIG. 5 graphically illustrates the loading efficiency of trehalose into human erythrocytic cells as a function of external trehalose concentration at respective temperatures of 4° C. and 37° C. Erythrocytic cells were exposed to trehalose for 18 hours at either 4° C. or 37° C. The trehalose concentration in the incubation medium varied between 230 mM and 1000 mM. Each incubation buffer contained trehalose (between 230 mM and 1000 mM) and 100 mOsm PBS pH 7.2. Increase in the trehalose concentration in the loading medium results in an increase in the sugar uptake, reaching about 100 mM cytoplasmic trehalose in erythrocytes incubated in 1000 mM trehalose and 100 mOsm PBS. At 4° C., the uptake was very limited, being about 25 mM. The trehalose intake was measured using anthrone assay and confirmed by high performance liquid chromatography. It is clear that there was substantial loading at 37° C., but not at 4° C. Furthermore, trehalose loading was not significant unless the extracellular cellular trehalose concentration gives a hyperosmotic pressure. Since intracellular osmolarity for erythrocytic cells is about 300 mOsm, it is clear that raising the extracellular osmolarity was required for more effective loading of trehalose.

### EXAMPLE 3

FIG. 6 graphically illustrates the fragility index of erythrocytic cells incubated overnight at respective temperatures of 4° C. and 37° C. in the presence of and as a function of increasing intracellular trehalose concentrations. The osmotic fragility index was generated by the extent of hemolysis as a function of the NaCl concentration. The erythrocytic cells that had been loaded in trehalose solutions (between 250 mM and 1000 mM) in 100 mOsm PBS were suspended in increasing concentrations of NaCl (between 50 and 600 mOsm NaCl). The percent hemolysis measured after resuspending the loaded cells in NaCl represents the fragility index. The data show that the erythrocytic cells were stable osmotically in trehalose media with concentrations between 250 mM and 800 mM trehalose at both 37° C. and 4° C. In 1000 mM trehalose at 37° C., there is a high increase in the fragility index suggesting that the cells were unstable in this medium (1000 mM trehalose in 100 mOsm PBS).

### EXAMPLE 4

FIG. 7 graphically illustrates trehalose uptake (i.e., intracellular trehalose mM) and hemolysis (i.e., % hemolysis) as a function of incubation temperature (° C.). The incubation temperature was varied between 4° C. and 37° C. The erythrocytic cells were incubated for 6 hours in 800 mM trehalose in 100 mOsm PBS pH 7.2. Between 4° C. and 30° C., the cytoplasmic trehalose was very low (between 1 and 4 mM). It was considerably increased (up to 35 mM cytoplasmic trehalose) during 6 hours incubation at 37° C.

### EXAMPLE 5

FIG. 8 graphically illustrates intracellular trehalose concentration (mM) as a function of the osmolarity of the washing buffer. Earlier morphological data showed that along with discoid erythrocytic cells, there is about 20% of cells with modified shape (spherocytes and schistocytes). The issue was what was the loading capacity of these cells and how much they contribute to the amount of trehalose that was to be detected. This issue was investigated by washing the trehalose loaded erythrocytic cells (loaded at 35° C. for 16 hours in 800 mM trehalose in 100 mOsm PBS pH 7.2) in buffers with different osmolarity (300 mOsm PBS or 900 mOsm PBS) and estimating the cytoplasmic sugar concentration. The loaded cells were washed with either 300 mOsm PBS pH 7.2 (which is the isotonic medium for erythrocytic cells) or 900 mOsm PBS pH 7.2 (which matches the tonicity of the loading medium). The data in FIG. 8 illustrated that there is a decrease in the intracellular sugar concentration suggesting that a fraction of the cells was lost during the washing procedure.

### EXAMPLE 6

FIG. 9 graphically illustrates % hemolysis of loaded cells as a function of time (hours) of incubation in 300 mOsm PBS pH 7.2. The erythrocytic cells were loaded in 700 mM trehalose in 100 mOsm PBS pH 7.2 at 35° C. for 16 hours. After the loading step, the cells were incubated in 300 mOsm PBS pH 7.2 between 1 and 25 hours. This buffer was used for washing the loaded cells and it was important to determine the stability of the loaded cells when they were suspended in this buffer. Cell stability was assessed by measuring the percent hemolysis over the course of 25 hours. The data show an increase in the percent hemolysis in the first 7 hours (from about 7% to about 25% hemolysis). Between 7 and 25 hours, there was only a small change in the percent hemolysis (from about 25% to about 30%).

### EXAMPLE 7

FIG. 10 graphically illustrates time course (incubation time, hours) of hemolysis(%) of trehalose-loaded cells as a function of the composition of the incubation buffer. Erythrocytic cells were loaded in 700 mM trehalose in 100 mOsm PBS pH 7.2 for 16 hours at 35° C. They were washed with 300 mOsm PBS pH 7.2 and were subsequently transferred to storage media with increasing trehalose concentrations (between 100 mM and 300 mM trehalose) added to 100 mOsm PBS pH 7.2. The percent hemolysis was measured over the course of 3 hours. The results show that the percent hemolysis is reduced with increase of trehalose concentration (from about 25% in 300 mOsm PBS with no trehalose, to about 4% in 300 mM trehalose in 100 mOsm PBS).

### EXAMPLE 8

FIG. 11 graphically illustrates time course (incubation time, hours) of hemolysis(%) of trehalose-loaded cells as a function of the composition of the incubation buffer, and illustrating that HES and albumin (HSA) do not have any detrimental effect on cell hemolysis during incubation. We tested the effect of 15% HES and 30% HES, and the effect of 2.5% albumin (HSA) and 5% albumin, as well as a combination of 2.5% albumin and 15% HES. The results show that a combination of the two compounds provides the lowest percent hemolysis of trehalose-loaded erythrocytic cells.

### EXAMPLE 9

FIG. 12 is a 40× picture of rehydrated erythrocytic cells having no intracellular trehalose prior to freeze-drying. These cells had not been loaded with trehalose, and were freeze-dried in 300 mM trehalose in 100 mOsm PBS, 15% HES and 2.5% albumin. Rehydration was done in 188 mM trehalose, 100 mOsm PBS, 15% HES and 2.5% albumin. FIG. 12 illustrates that when the cells are not loaded with trehalose, and are freeze-dried and rehydrated, there are mostly cells with modified shape (spherocytes) and cell debris.

FIG. 13 is a 40× picture of rehydrated erythrocytic cells having 3 mM intracellular trehalose, after initially trehalose-loading the cells in 400 mM trehalose in 100 mOsm PBS at 35° C. for 16 hours and freeze-drying the cells in 300 mM trehalose/100 mOsm PBS, 15% HES and 2.5% HSA. Rehydration was done in 188 mM trehalose, 100 mOsm PBS, 15% HES and 2.5% albumin. FIG. 13 displays erythrocytic cells containing 3 mM cytoplasmic trehalose, freeze-dried and rehydrated. Cells with biconcave shape were seen, as well as cells with modified morphology, suggesting that trehalose loading of erythrocytic cells provide higher number of cells with preserved morphology.

FIG. 14 is a 40× picture of rehydrated erythrocytic cells having 60 mM intracellular trehalose, after initially trehalose-loading in 800 mM trehalose in 100 mOsm PBS at 35° C. for 16 hours and freeze-drying the cells in 300 mM trehalose/100 mOsm PBS, 15% HES and 2.5% HSA. Rehydration was done in 188 mM trehalose, 100 mOsm PBS, 15% HES and 2.5% albumin. FIG. 14 shows erythrocytic cells containing 60 mM cytoplasmic trehalose, freeze-dried and rehydrated. The typical biconcave shape and cell integrity appear well preserved.

FIG. 15 is a 100× picture of rehydrated erythrocytic cells having 60 mM intracellular trehalose, after initially trehalose-loading in 800 mM trehalose in 100 mOsm PBS at 35° C. for 16 hours and freeze-drying the cells in 300 mM trehalose/100 mOsm PBS, 15% HES and 2.5% HSA. Rehydration was done in 188 mM trehalose, 100 mOsm PBS, 15% HES and 2.5% albumin. FIG. 15 shows erythrocytic cells containing 60 mM cytoplasmic trehalose, freeze-dried and rehydrated at higher magnification (100×) confirming that the cell shape is mostly biconcave and cell integrity is preserved.

### EXAMPLE 10

FIG. 16 is a graph of hemolysis(%) of trehalose loaded, freeze-dried and rehydrated erythrocytic cells as a function of intracellular trehalose concentration (mM), graphically illustrating the effect of cytoplasmic trehalose on the survival of rehydrated erythrocytic cells. The cells were loaded in media with different trehalose concentration (400, 500, 600, 700 and 800 mM trehalose) in 100 mOsm PBS pH 7.2. The cells were freeze-dried in 300 mM trehalose in 100 mOsm PBS, 15% HES and 2.54% HSA and rehydrated in a buffer containing 188 mM trehalose, 15% HES and 2.5% HSA. FIG. 16 shows that higher extent of trehalose loading of erythrocytic cells confers higher percent of survival, assessed by the percent hemolysis of the rehydrated cells.

### EXAMPLE 11

FIG. 17 is a graph of mean corpuscular hemoglobin of trehalose-loaded, freeze-dried and rehydrated erythrocytic cells as a function of intracellular trehalose concentration (mM), graphically illustrating that as the concentration of intracellular trehalose increases for rehydrated erythrocytic cells, the mean corpuscular hemoglobin (the amount of hemoglobin found in intact erythrocytic cells) also increases for rehydrated erythrocytic cells. The cells had been loaded in media with different trehalose concentration (400 mM trehalose, 500 mM trehalose, 600 mM trehalose, 700 mM trehalose and 800 mm trehalose) in 100 mOsm PBS pH 7.2. The cells were freeze-dried in 300 mM trehalose in 100 mOsm PBS, 15% HES and 2.54% HSA and rehydrated in a buffer containing 188 mM trehalose, 15% HES and 2.5% HSA. The data demonstrate that higher cytoplasmic trehalose increases the amount of mean corpuscular hemoglobin in rehydrated cells (from about 7 pg in cells loaded in 400 mM trehalose to about 14 pg in cells loaded in 800 mM trehalose).

### EXAMPLE 12

The following protocol provided significant survival of freeze-dried and rehydrated erythrocytic cells. Loading buffer comprised 800 mM trehalose in a salt solution of 100 mOsm PBS. The incubation time was 16 hours at a temperature of about 35° C. After the cells were loaded, they were subsequently washed in a washing buffer comprising 300 mM in a salt solution of 100 mOsm PBS. Within 3 hours after washing the loaded cells, the wash loaded cells were freeze-dried in freeze-drying buffer comprising about 300 mM trehalose, about 100 mOsm PBS, about 2.5% by wt. HSA, and about 15% by wt. HES. After the freeze-drying procedure the cells had about 75 mM trehalose, about 25 mOsm PBS, about 0.6% by wt. HSA and about 4.0% by wt. HES left in the cells. The freeze-dried cells were then reconstituted at about 37° C. for about 10 minutes in a rehydration buffer comprising about 188 mM trehalose, about 100 mOsm PBS, about 2.5% by wt. HSA and about 15.0% by wt. HES. After rehydration, less than about 5% of the cells lysed.

### EXAMPLE 13

FIG. 24 shows the trehalose uptake by the erythrocytic cells monitored in dependence of time of incubation. The cells were incubated in a medium composed of 800 mM trehalose, 100 mOsm ADSOL and 6.6 mM K-phosphate. 100 mOsm ADSOL contains 24 mM glucose, 0.43 mM adenine, 33.3 mM NaCl and 8.9 mM mannitol. Four different temperatures of incubation were used: 4° C., 23° C., 37° C. and 41° C. The data show an increase of the uptake of trehalose by the erythrocytic cells with an increase of temperature. At 4° C., the uptake was the lowest and only slightly increased during 16 hours of incubation (10 mM). At 23° C., the uptake was higher than at 4° C. and increased with increase of the time of incubation reaching 22 mM intracellular trehalose. At 37° C., the highest uptake of trehalose by the erythrocytic cells was observed within 16 hours (60 mM). At 41° C., the uptake was slightly higher than that observed at 37° C. within 6 hours. Since the percent hemolysis (corresponding to cells that have lost their intracellular hemoglobin) was higher than that observed at the other temperatures, we did not continue the incubation of the erythrocytic cells at 41° C. for more than 6 hours.

FIG. 25 presents the percent hemolysis of erythrocytic cells incubated in 800 mM trehalose, 100 mOsm ADSOL and 6.6 mM K-phosphate at 4° C., 23° C., 37° C. and 41° C. At 4° C., the percent hemolysis is low (2%) during 9 hours of incubation of erythrocytic cells in 800 mM trehalose, 100 mOsm ADSOL and 6.6 mM K-phosphate. At 23° C., the percent hemolysis increases to 7% during 6 hours of incubation. At 37° C., between 10 and 12% hemolysis is observed during incubation of erythrocytic cells between 6 and 9 hours. These results demonstrate that between 6 and 9 hours of incubation of erythrocytic cells in 800 mM trehalose, 100 mOsm ADSOL and 6.6 mM K-phosphate, the percent hemolysis is maintained low (between 10 and 12%) (FIG. 25) and the trehalose uptake at these conditions is between 23 and 55 mM intracellular trehalose (FIG. 24). Such intracellular concentration of trehalose provides high survival of erythrocytic cells after freeze-drying and rehydration, therefore these conditions (incubation in trehalose medium between 6 and 9 hours at 37° C.) can be used for loading erythrocytic cells.

### EXAMPLE 14

In order to specify more precisely which conditions are optimal for incubation of erythrocytic cells in trehalose containing medium, we studied changes in the level of two important metabolites, adenosine-3-phosphate (ATP) and 2,3-diphosphoglycerate (2,3-DPG) during incubation at either 4° C. or 37° C. FIG. 26 presents changes in the level of ATP during incubation of erythrocytic cells in either 800 mM trehalose, 100 mOsm ADSOL and 6.6 mM K-phosphate or in 460 mOsm ADSOL at 4° C. and at 37° C. 100 mOsm ADSOL medium contains 24 mM glucose, 0.43 mM adenine, 33.3 mM NaCl and 8.9 mM mannitol. 460 mOsm ADSOL medium contains 111 mM glucose, 2 mM adenine, 154 mM NaCl and 41 mM mannitol and is the medium used by the blood banks for storing erythrocytic cells. During incubation of erythrocytic cells at 4° C. in either 800 mM trehalose and 100 mM ADSOL or in 460 mOsm ADSOL (for control cells), the level of ATP is maintained high during 24 hours of incubation (between 4.5 and 4.2 μmol/g Hb for the control cells, and between 4 and 3.1 μmol/g Hb in cells incubated in trehalose containing medium) and is very similar to the one of freshly isolated erythrocytic cells (between 3.65 and 4.45 μmol/g Hb). At 37° C., the level of ATP does not decrease during 16 hours of incubation in either 800 mM trehalose, 100 mOsm ADSOL and 6.6 mM K-phosphate or in 460 mM ADSOL.

At 4° C., the level of 2,3-DPG of erythrocytic cells incubated in either 800 mM trehalose, 100 mOsm ADSOL and 6.6 mM K-phosphate or in 460 mOsm ADSOL remains high (between 10.8 and 9 μmol/g Hb in control cells, and between 9.5 and 7 μmol/g Hb in cells incubated in trehalose medium) during 24 hours incubation and is close to that of freshly isolated erythrocytic cells. The normal level of 2,3-DPG in freshly isolated cells is 13±3 μmol/g Hb. At 37° C., however, the level of 2,3-DPG decreases in erythrocytic cells incubated in 460 mOsm ADSOL during the first 7 hours. In contrast, in the presence of 800 mM trehalose, 100 mOsm ADSOL and 6.6 mM K-phosphate, the level of 2,3-DPG is high during 7 hours of incubation. These data demonstrate that 7 hours incubation in 800 mM trehalose, 100 mOsm ADSOL and 6.6 mM K-phosphate medium at 37° C. is suitable for incubation erythrocytic cells, since they maintain high levels of ATP and 2,3-DPG.

### CONCLUSION

Embodiments of the present invention provide that trehalose, a sugar found at high concentrations in organisms that normally survive dehydration, may be used to preserve biological structures in the dry state. Cells may be loaded with trehalose under the previously specified conditions, and the loaded cells can be freeze dried with excellent recovery.

While the present invention has been described herein with reference to particular embodiments thereof, a latitude of modification, various changes and substitutions are intended in the foregoing disclosure, and it will be appreciated that in some instances some features of the invention will be employed without a corresponding use of other features without departing from the scope and spirit of the invention as set forth. Therefore, many modifications may be made to adapt a particular situation or material to the teachings of the invention without departing from the essential scope and spirit of the present invention. It is intended that the invention not be limited to the particular embodiment disclosed as the best mode contemplated for carrying out this invention, but that the invention will include all embodiments and equivalents falling within the scope of the appended claims.

1. A method for loading a biological sample with a solute comprising:
disposing a biological sample into a solute solution comprising a solute and a chemical selected from the group consisting of a monosaccharide, a monosaccharide polyol, a cell metabolite-controlling agent, a salt, and mixtures thereof; and
incubating the biological sample in the solute solution while maintaining a positive solute concentration increase (mM) to incubation (hours) loading gradient during incubating to load the biological sample with the solute.

2. The method of claim 1 wherein said incubating comprises incubating the biological sample at a temperature ranging from about 35° C. to about 39° C. and from about 6 hours to about 10 hours.

3. The method of claim 1 wherein said monosaccharide polyol comprises mannitol.

4. The method of claim 1 wherein said cell metabolite-controlling agent comprises adenine.

5. The method of claim 1 wherein said monosaccharide comprises glucose.

6. The method of claim 1 wherein said metabolite comprises ATP.

7. The method of claim 1 wherein said biological sample comprises a mammalian biological sample.

8. The method of claim 1 wherein said solute solution additionally comprises a buffering salt compound.

9. A biological sample produced in accordance with the method of claim 1.

10. A method for maintaining a viability level of a metabolite in a biological sample comprising:
disposing a biological sample into a solute solution comprising a solute and a chemical selected from the group consisting of a monosaccharide, a monosaccharide polyol, a cell metabolite-controlling agent, a salt, and mixtures thereof; and
incubating the biological sample in the solute solution while maintaining a viability level of a metabolite in the biological sample.

11. The method of claim 12 additionally comprising maintaining a positive gradient of metabolite level during said incubating.

12. The method of claim 10 wherein said maintaining a viability level of a metabolite comprises preventing a decrease in a metabolite level of the biological sample.

13. The method of claim 10 wherein said metabolite is selected from the group consisting of ATP, 2,3-DPG and mixtures thereof.

14. The method of claim 10 wherein said incubating the biological sample in the solute solution comprises incubating at a temperature ranging from about 35° C. to about 39° C. for a period of time ranging from about 4 hours to about 8 hours.

15. A method for maintaining a viability level of a metabolite in a biological sample comprising:
disposing a biological sample into a solute solution comprising a solute and a chemical selected from the group consisting of a monosaccharide, a monosaccharide polyol, a cell metabolite-controlling agent, a salt, and mixtures thereof; and
incubating the biological sample in the solute solution while maintaining a maintaining a positive gradient of change in a level of a metabolite to change in duration of incubation.

16. The method of claim 15 wherein said metabolite comprises ATP.
